(19) United States
(12) Patent Application Publication
(10) Pub. No.: US 2026/0151149 A1
(43) Pub. Date: Jun. 4, 2026

(54) SYSTEMS FOR DELIVERING HIGH INTENSITY FOCUSED ULTRASOUND INCLUDING ROBOTIC MANIPULATORS, AND DEVICES AND METHODS THEREOF

(71) Applicant: Petal Surgical, Inc., Foster City, CA (US)

(72) Inventor: Roman DEVENGENZO, San Jose, CA (US)

(21) Appl. No.: 19/458,437

(22) Filed: Jan. 23, 2026

Related U.S. Application Data
(63) Continuation of application No. PCT/US2025/043796, filed on Aug. 27, 2025.
(60) Provisional application No. 63/687,745, filed on Aug. 27, 2024.
(60) Provisional application No. 63/715,744, filed on Nov. 4, 2024.

(51) Int. Cl. A61B 17/22; A61B 17/00; A61B 34/30

(52) U.S. Cl. CPC A61B 17/2202; A61B 34/30; A61B 2017/00699; A61B 2017/22008; A61B 2017/22028

(57) ABSTRACT

Systems, devices, and methods described herein relate to non-invasively treating tissue using high-intensity focused ultrasound (HIFU), including boiling histotripsy. In some embodiments, HIFU systems described herein include robotic systems including positioning arms and/or other positioning devices, which can be configured to provide for gross and micro adjustments and movements of one or more end effector assemblies. The end effector assemblies can include treatment device configured to treat one or more regions of tissue.

### CROSS-REFERENCE TO RELATED APPLICATIONS

This application is a Continuation Application of International Application No. PCT/US 2025/043796, filed Aug. 27, 2025, entitled “SYSTEMS FOR DELIVERING HIGH INTENSITY FOCUSED ULTRASOUND INCLUDING ROBOTIC MANIPULATORS, AND DEVICES AND METHODS THEREOF,” which claims priority to and the benefit of U.S. Provisional Ser. No. 63/687,745 , filed Aug. 27, 2024, entitled “HISTOTRIPSY SYSTEMS INCLUDING ROBOTIC MANIPULATORS, AND DEVICES AND METHODS THEREOF,” and U.S. Provisional Ser. No. 63/715,744 , filed Nov. 4, 2024, entitled “HISTOTRIPSY SYSTEMS INCLUDING ROBOTIC MANIPULATORS, AND DEVICES AND METHODS THEREOF,” the entire contents of each of which are hereby incorporated by reference in their entireties.

### TECHNICAL FIELD

The embodiments described herein relate generally to systems, devices, and methods for treating tissue, including systems, devices, and methods of treating tissue using boiling histotripsy (BH) or acoustic jet liquefaction.

### BACKGROUND

During some procedures, it may be beneficial for surgery to be minimally invasive to reduce the likelihood of bleeding complications and/or infection resulting from the procedure. Once such minimally invasive procedure can include using high-intensity focused ultrasound (HIFU) to cause mechanical and/or thermal effects in tissue, e.g., to disrupt and treat targeted tissue such as a tumor. With boiling histotripsy, bursts or pulses of HIFU can form positive pressure (e.g., shock waves or shock fronts) that leads to heat deposition through absorption of the shocks. This in turn can lead to the generation of vapor bubbles, which can interact with remaining cycles of the HIFU bursts to lead to tissue fractionation in a targeted region (e.g., a focal region).

Boiling histotripsy can be employed as noninvasive means to ablate unwanted tissue, including but not limited to treatment for benign or malignant tumors, benign prostatic hyperplasia (BPH), deep vein thrombosis, and congenital heart defects. Boiling histotripsy treatments can cause mechanical disruption of tissue with well-demarcated regions of mechanically emulsified treatment volumes that have little remaining cellular integrity. For certain medical applications, tissue emulsification may be more favorable than thermal damage because it lends itself to tissue selectivity allowing for the preservation of healthy tissue and produces liquefied volumes that can be more easily removed or absorbed by the body than thermally coagulated solid volumes.

There is a need for systems, devices, and methods of performing boiling histotripsy that are more efficient and supports clinicians to reduce workload for the clinician.

### SUMMARY

The present disclosure is directed toward methods and systems for non-invasively treating tissue using HIFU. Systems, devices, and methods can include robotic systems and/or components that are configured to manipulate (e.g., operate, move, etc.) one or more components of a BH system to treat tissue.

In some embodiments, an apparatus includes an end effector assembly including: a treatment device including an array of ultrasound transducers configured to generate pulses of pressure waves that are configured to travel through an interfacing fluid and into tissue to induce boiling histotripsy at a focal volume, an imaging device configured to capture a view of a three-dimensional (3D) target area of a patient, and a positioning device configured to move the treatment device in at least three degrees-of-freedom (DOFs) to cause the focal volume to move throughout the 3D target area to treat the 3D target area using boiling histotripsy; and a robotic positioning arm coupled to the end effector assembly, the robotic positioning arm including a plurality of joints and segments that are configured to move the end effector assembly in at least three degrees-of-freedom to position the treatment device near the 3D target area such that the treatment device can treat the 3D target area.

In some embodiments, an apparatus includes an end effector assembly including: a treatment device including an array of ultrasound transducers configured to generate pulses of pressure waves that are configured to travel through an interfacing fluid and into tissue to induce boiling histotripsy at a focal volume, a positioning device configured to move the treatment device in at least three degrees-of-freedom to cause the focal volume to move throughout a three-dimensional (3D) target area of a patient to treat the 3D target area using boiling histotripsy, and a robotic positioning arm coupled to the end effector assembly, the robotic positioning arm including a plurality of joints and segments that are configured to move the end effector assembly in at least three degrees-of-freedom to position the treatment device near the 3D target area and to compensate for gross movements such that the treatment device can treat the 3D target area.

In some embodiments, an apparatus includes an end effector assembly including: an instrument configured to be inserted into tissue of a patient; a treatment device disposed on a distal end portion of the instrument, the treatment device including an array of ultrasound transducers configured to generate pulses of pressure waves that are configured to travel through an interfacing fluid and into the tissue to induce boiling histotripsy at a focal volume, and a positioning device configured to move the treatment device in at least three degrees-of-freedom (DOFs) to cause the focal volume to move throughout a three-dimensional (3D) target area of the patient to treat the 3D target area using boiling histotripsy; a robotic positioning arm coupled to the end effector assembly, the robotic positioning arm including a plurality of joints and segments that are configured to move the end effector assembly in at least three degrees-of-freedom to position the treatment device near the 3D target area such that the treatment device can treat the 3D target area; and a sterile barrier configured to cover at least a portion of the robotic positioning arm while allowing for coupling of at least the instrument to the robotic positioning arm.

### BRIEF DESCRIPTION OF THE DRAWINGS

FIG. 1 schematically depicts a HIFU system configured to induce boiling histotripsy or acoustic jet liquefaction, according to embodiments.

FIG. 2A schematically depicts a controller of a HIFU system, according to embodiments.

FIG. 2B schematically depicts a histotripsy device of a HIFU system, according to embodiments.

FIG. 2C schematically depicts a robotic system of a HIFU system, according to embodiments.

FIG. 3A schematically depicts a configuration of a histotripsy device, operating to effect mechanical and/or thermal changes in tissue, according to embodiments.

FIG. 3B depicts a graph showing a HIFU voltage pulse, according to embodiments.

FIG. 3C schematically depicts the mechanical and/or thermal changes in tissue, in response to application of HIFU, according to embodiments.

FIGS. 4A and 4B depict an example robotic arm of a robotic surgical system configured to control movements of a distal structure of a HIFU system, according to embodiments.

FIGS. 5A and 5B depict another example robotic arm of a robotic surgical system configured to control movements of a distal structure of a HIFU system, according to embodiments.

FIGS. 6A and 6B depict another example robotic arm of a robotic surgical system configured to control movements of a distal structure of a HIFU system, according to embodiments.

FIGS. 7A and 7B depict another example robotic arm of a robotic surgical system configured to control movements of a distal structure of a HIFU system, according to embodiments.

FIGS. 8A and 8B depict another example robotic arm of a robotic surgical system configured to control movements of a distal structure of a HIFU system, according to embodiments.

FIGS. 9A and 9B depict another example robotic arm of a robotic surgical system configured to control movements of a distal structure of a HIFU system, according to embodiments.

FIGS. 10A and 10B depict another example robotic arm of a robotic surgical system configured to control movements of a distal structure of a HIFU system, according to embodiments.

FIGS. 11A-11E depict clinical positioning and orienting of a distal structure of a HIFU system using a robotic arm, according to embodiments.

FIG. 11F depicts various degrees-of-freedom (DOFs) of the robotic arm of FIGS. 11A-11E, according to embodiments.

FIG. 12A depicts various DOFs of a distal structure of a HIFU system, according to embodiments.

FIGS. 12B-12D depict movements of a HIFU treatment head of a HIFU system, according to embodiments.

FIG. 13 depicts hardware-based respiration compensation of a HIFU system, according to embodiments.

FIGS. 14A-14C depict different HIFU systems, designed for different applications, according to embodiments.

FIG. 15 depicts an HIFU system, according to embodiments.

FIG. 16 depicts a HIFU treatment device, according to embodiments.

FIG. 17 depicts a robotic arm of a robotic surgical system coupled to a robot cart, according to an embodiment.

FIGS. 18A-18B depict another example robotic arm of a robotic surgical system configured to control movements of a distal structure of a HIFU system, according to embodiments.

FIGS. 19A-19B depict another example robotic arm of a robotic surgical system configured to control movements of a distal structure of a HIFU system, according to embodiments.

FIG. 20 depicts various DOFs of a robotic arm, according to an embodiment.

FIG. 21 depicts a center of motion of a robotic arm, according to an embodiment.

FIGS. 22A-22B depict another example robotic arm of a robotic surgical system configured to control movements of a distal structure of a HIFU system, according to embodiments.

FIGS. 23A-23B depict different HIFU systems, designed for different applications, according to embodiments.

FIG. 24 depicts the HIFU system of FIGS. 22A-22B in an orientation, according to embodiments.

FIG. 25 depicts different applications for HIFU systems, according to embodiments.

FIGS. 26A-26B schematically depict a robotic system configured to position a HIFU system, according to embodiments.

Optional components and/or elements in the figures are shown in dashed lines, and/or described as such in the paragraphs that follow.

### DETAILED DESCRIPTION

### I. Overview of Systems and Devices

The embodiments described herein relate generally to systems, devices, and methods for treating tissue of a patient using HIFU to induce boiling histotripsy or acoustic jet liquefaction. Boiling histotripsy or acoustic jet liquefaction is a minimally invasive, incisionless procedure that can be applied to tissue to liquefy the tissue in the treatment area. The liquified tissue can be absorbed by surrounding tissue, passed out by the body, or can be removed for analysis (e.g., biopsy, etc.). In some embodiments, a robotic system can be used for positioning a histotripsy device to target a treatment area (e.g., target area, anatomical region of interest, etc.). The treatment area can be any size and/or shape, and in some embodiments, can be sized and/or shaped to a particular individual and/or for a particular indication or target anatomy. In some embodiments, the robotic system may move along a predefined treatment path to treat a larger treatment area. In some embodiments, the robotic system can be utilized for positioning the histotripsy device to target multiple clinically relevant sections customized in both size and shape for a planned treatment. In some embodiments, various parameters of a HIFU waveform (e.g., duty cycle, pulse repetition rate, amplitude, frequency, etc.) and/or movement of a HIFU transducer array can be adjusted during treatment, e.g., by a controller and/or robotic system.

In some embodiments, the systems, devices, and methods described herein support pre-operative and/or intra-operative clinical decisions and/or treatment planning. For example, to support clinical decisions, systems, devices, and methods described herein can be configured to update or modify a treatment plan based on extraction of treated tissue and/or intra-operative imaging (e.g., using imaging ultrasound). In some embodiments, the treatment plan can be updated as a procedure continues, thus increasing the effectiveness of the procedure. In some embodiments, the system, devices, and methods described herein can include visualizations and/or interfaces for communicating information to medical professionals to aid in clinical decision making.

In some embodiments, an imaging and tracking system can be used to track the components of a histotripsy system relative to each other and to patient anatomy. This may allow for live treatment planning by a medical professional. For example, the medical professional can be configured to adjust the operation of a robotic device during HIFU treatment, to adjust HIFU waveform parameters, to pause treatment, to adjust the placement or location of one or more components of the HIFU system, among other things. In some embodiments, the systems, devices, and methods described herein can be used to analyze a treatment site and determine whether one or more nearby anatomical structures (e.g., nerves) may need to be moved or avoided for a procedure to proceed safely. For example, in spine procedures, systems, devices, and methods described herein can image around a treatment site and determine whether one or more nerves or other anatomical structures may need to be avoided and/or moved to enable treatment of the treatment site. In some embodiments, systems, devices, and methods described herein can provide percutaneous tools or instruments for moving patient anatomical structure, such as a nerve. While certain embodiments described herein are described with reference to specific anatomical regions of interest or specific treatments, the devices, systems, and methods described herein can configured for use with other procedures and treatments.

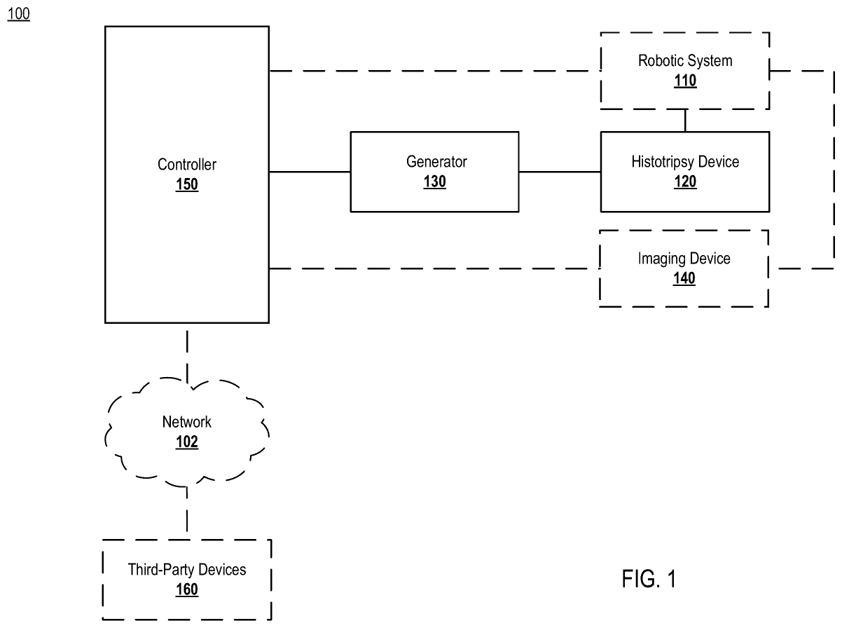

FIG. 1 schematically depicts a HIFU or histotripsy system 100, according to embodiments. The system 100 is configured to apply HIFU to induce boiling histotripsy or acoustic jet liquefaction in tissue. The histotripsy system 100 includes a histotripsy device 120 (e.g., treatment device or HIFU treatment device), a generator 130, and a controller 150. In some embodiments, the histotripsy system 100 optionally includes a robotic system 110, an imaging device 140, and one or more third-party devices 160. Any combination of the generator 130, the histotripsy device 120, the imaging device 140, the robotic system 110, and/or the controller 150 can be parts of a single device or a plurality of devices.

In embodiments, the histotripsy system 100 can be configured to treat herniated discs in the spine. In embodiments, the histotripsy system 100 can be configured to treat benign tumors, cysts, and/or fibroids, e.g., in the breast, uterine wall, or other anatomy, and/or undesirable tissue growth. In embodiments, the histotripsy system 100 can be configured to treat endometriosis. In some embodiments, the histotripsy system 100 can be configured to treat malignant tumors. In some embodiments, the histotripsy system 100 can be configured to treat bacterial growths. In some embodiments, the histotripsy system 100 can be configured to treat blood clots. The generator 130 is configured to generate and supply energy to the histotripsy device 120. In some embodiments, the generator 130 is configured to generate a pulse waveform. The generator 130 may include its own power source (e.g., battery, batteries, etc.) or receive power from an external power source (e.g., grid power, clinic power, generator, etc.). In some embodiments, the generator 130 may include one or more inputs (e.g., button, switch, dial, touchscreen, or other user interface, whether directly or remotely coupled thereto) for adjusting the energy (e.g., pulse waveforms) generated by the generator 130. In some embodiments, the generator 130 can be a function generator such as, for example, an Agilent 33250A function generator. In some embodiments, the generator 130 can include electrical components that are configured to store energy. For example, the generator 130 can include capacitors that are configured to store energy from a power supply. The generator 130 can be operatively coupled to the controller 150 and the histotripsy device 120, and receive signals from one or both of the controller 150 and the histotripsy device 120. In some embodiments, the generator 130 may deliver a pulse waveform to the histotripsy device in response to receiving a signal indicating that delivering energy to the histotripsy device 120 is desired. In some embodiments, the generator 130 may include one or more electrical components (e.g., electrical conduit, electrical port, etc.) that allows the histotripsy device to operably couple to the generator 130. In some embodiments, the generator 130 is configured to deliver voltage waveforms to the histotripsy device 120 such that the histotripsy device 120 has an output power of between about 300 Watts and about 4,000 Watts, including all sub-ranges and values therebetween.

The histotripsy device 120 is configured to convert the energy generated by the generator 130 into focused ultrasonic waves for histotripsy. For example, the histotripsy device 120 can be configured to receive a pulse waveform from the generator 130, and to generate a pulsatile wavefront of ultrasound radiation. In embodiments, the histotripsy device 120 can include a transducer array, having a plurality of transducers that can generate HIFU waves. The HIFU waves can be configured to converge at a focal point or focal volume, e.g., to generate a lesion. The focal point or focal volume can be located in a target area selected for treatment. Focusing the ultrasound allows for the HIFU waves to deliver enough energy to the target area to allow for BH. BH includes the heating and formation of bubbles in tissue. In particular, pulsatile wavefronts generated by HIFU transducers can be configured to produce vapor bubbles at a focal point or focal volume by heating up the tissue, and to interact with those bubbles to produce cavitation of the bubbles.

The histotripsy device 120 can be positioned by a physician and/or by the robotic system 110. In some embodiments, during treatment, the position of the histotripsy device 110 can be adjusted or moved by the robotic system 110, as further described below. In some embodiments, the histotripsy device 120 can include at least one input. For example, the histotripsy device 120 can include an activation button that, when actuated, can generate a signal to activate the generator 130 and/or activate delivery of a pulse waveform to the histotripsy device 120. In some embodiments, the histotripsy device 120 can be coupled to a robotic system 110, and the robotic system 110 can be configured to signal the generator 130 to deliver pulse waveforms to the histotripsy device 120 during operation.

The system 100 can be configured to implement a pulsing protocol that takes into account various parameters including ultrasound frequency, pulse repetition frequency (PRF), pulse length, duty cycle, pressure amplitude, etc. For example, the histotripsy device 120 can be configured to have an output frequency of between about 1 MHz and about 3 MHz, including all sub-ranges and values therebetween, and an output power of between about 300 Watts and about 4,000 Watts, including all sub-ranges and values therebetween. The histotripsy device 120 can generate a pulsatile wavefront that includes a plurality of waves formed into a HIFU pulse. In some embodiments, each HIFU pulse has a pulse duration of between about 1 and about 30 milliseconds, including all sub-ranges and values therebetween. The system 100, via control of the generator 130, can be configured to produce the pulsatile wavefront for the pulse duration, followed by a pause, before initiating a subsequent pulse, thereby producing the HIFU pulses at a set PRF. In some embodiments, the PRF of the HIFU pulses can be about 0.5 Hz to about 10 Hz, including all sub-ranges and values therebetween. Having lower pulse repetition frequencies (e.g., less than about 20 Hz) can increase the efficiency of an operation, e.g., by firing and moving the treatment head to fire again and then repeating, vs. having to stay in one location for several seconds before being able to move.

In some embodiments, for inducing boiling histotripsy, the ultrasonic waves can be configured to have a peak positive pressure amplitude received at the treatment focus or focal point (e.g., a small volume of focus or focal volume) of greater than about 60 MPa, greater than about 70 MPa, or greater than about 80 MPa, including, for example, between about 70 and about 120 MPa (inclusive of all values and sub-ranges therebetween). The waves may be configured to have a peak negative pressure received at the treatment focus of greater than −15 MPa, greater than −10 MPa, or greater than −5 MPa, including, for example, between about −15 MPa and about −10 MPa (inclusive of all values and sub-ranges therebetween). The waves can be configured to have pulses of between about 1 and about 20 ms (inclusive of all values and sub-ranges therebetween). The waves can be configured to have a pulsing frequency or PRF of less than about 20 Hz, less than about 15 Hz, less than about 10 Hz, or less than about 5 Hz, including, for example, between about 10 and about 20 Hz (inclusive of all values and sub-ranges therebetween). In some embodiments, for inducing cavitation cloud histotripsy, the ultrasonic waves can be configured to have a peak negative pressure amplitude received at the treatment focus or focal point (e.g., a small volume of focus or focal volume) of greater than about −35 MPa, greater than about −30 MPa, or greater than about −25 MPa, including, for example, between about −35 MPa and −25 MPa (inclusive of all values and sub-ranges therebetween). The waves can be configured to have pulses of between about 1 and about 5 μs (inclusive of all values and sub-ranges therebetween). The waves can be configured to have a pulsing frequency or PRF of greater than about 100 Hz, including, for example, between about 100 and about 150 Hz (inclusive of all values and sub-ranges therebetween).

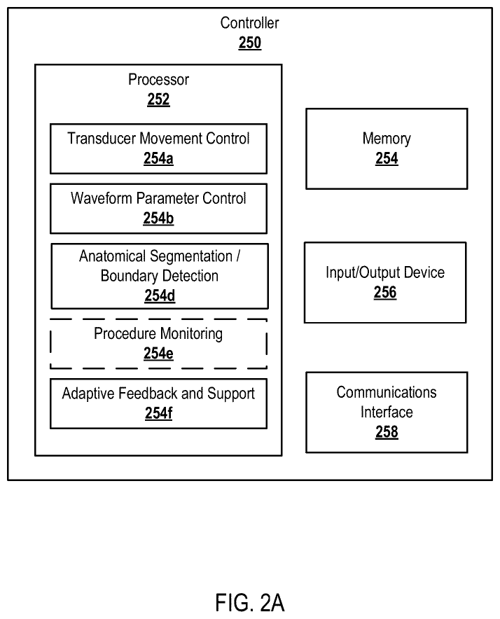

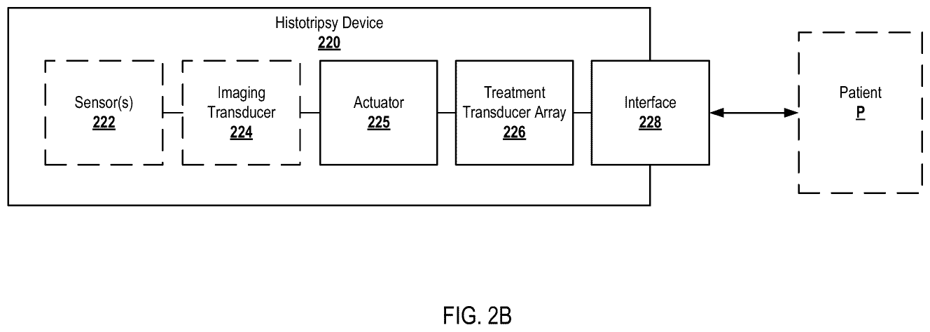

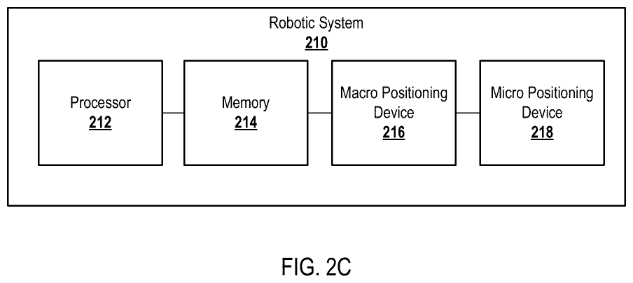

In some embodiments, the pulsing protocol associated with the HIFU treatment can be adjusted, e.g., based on monitoring of tissue and/or signals associated with the HIFU treatment. For example, the power output, the PRF, the duty cycle, etc. can be adjusted by adjusting the output from the generator 130. In some embodiments, the histotripsy device 120 can include one or more sensor(s) for measuring characteristics of the histotripsy device 120 (e.g., power output, energy usage, orientation, etc.). In some embodiments, the one or more sensors can be configured to measure characteristic of the tissue (e.g., temperature, size of lesion, shape of lesion, etc.). In some embodiments, the histotripsy device 120 can include or be coupled to an imaging transducer (e.g., of an imaging device 140). The imaging transducers can be configured to image the target area to allow for monitoring and/or visualization of the target area. The histotripsy device 120 is described in further detail in reference to FIG. 2B.

The robotic system 110 is configured to robotically position the histotripsy device 120 relative to a patient such that the ultrasonic waves generated by the histotripsy device 120 are directed toward the target area. In some embodiments, the robotic system 110 is communicatively coupled to the controller 150, which may be configured to command the operation of the robotic system 110. For example, the controller 150 may command the robotic system 110 to orient and/or move the histotripsy device 120 relative to the patient anatomy to generate one or more lesions. The robotic system 110 may include at least one arm and/or at least one joint that can translate and/or rotate the histotripsy device 120 along or about at least one axis. In some embodiments, the robotic system 110 can be operated by an operator (e.g., medical professional, surgical professional, ultrasound technician, etc.) or can be operated automatically by the controller 150. The robotic system 110 is described in further detail in reference to FIG. 2C.

In some embodiments, the system 100 can include an imaging device 140. In some embodiments, the imaging device 140 is configured to capture image data of a patient's anatomy and/or other devices nearby, prior to and/or during a HIFU treatment. For example, the imaging device can obtain pre-operative image data of an anatomical region of interest, obtain an intra-operative view of the anatomical region of interest, track the position of an intra-operative device (e.g., robotic device 110, histotripsy device 120, surgical device, etc.), and/or the like. In some embodiments, the histotripsy system 100 can include more than one imaging device 140. For example, the histotripsy system 100 can include an imaging device 140 for obtaining pre-operative image data, an ultrasound imaging device 140 for obtaining an intra-operative view, and/or an imaging device 140 for tracking the position of the intra-operative devices. In some embodiments, the imaging device 140 is statically positioned (e.g., focused on the target area, focused on an operating room, focused on the robotic system 110, etc.). In some embodiments, the imaging device 140 may be repositionable manually (e.g., by an operator) or by a robotic system (similar to the robotic system 110). In some embodiments, the imaging device 140 is communicably and/or operably coupled to controller 150. The imaging device 140 may be configured to receive commands from the controller 150 and/or to send image data to the controller 150. In some embodiments, the image data sent to the controller 150 can be used by the controller 150 to monitor temperature of the treated tissue, the boiling activity of the treated tissue, and/or other characteristics of the treated tissue. In some embodiments, the image data sent to the controller 150 can be used by the controller 150 to register pre-operative image data with intra-operative views of the treatment region. In some embodiments, the image data sent to the controller 150 can be used by the controller 150 to track one or more instruments, components, etc. in a working space.

The controller 150 is configured to control the operation of and/or communicate with the generator 130, the histotripsy device 120, the robotic system 110, and/or the imaging device 140. In some embodiments, the controller 150 is configured to process and/or analyze data received form the generator 130, the robotic system 110, and/or the imaging device 140. The controller 150 can be configured to modify the ultrasonic waves emitted by the histotripsy device 120, e.g., by modifying the power, voltage, pulse parameters, etc. of the generator 130. For example, the controller 150 can operate the generator 130 and the histotripsy 120 at a predetermined duty cycle. Additionally, or alternatively, the controller 150 may determine a treatment path and operate the robotic system 110 to position/orient the histotripsy device 120 to deliver treatment along the treatment path. In some embodiments, the controller 150 is configured to receive imaging data from the imaging device 140. In some embodiments, the controller 150 can generate a visualization of the position of at least one of the robotic system 110, the histotripsy device 120, and/or the anatomy of the patient based on the image data received form the imaging device 140. In some embodiments, the controller 150 can generate recommendations for a physician, e.g., based on data received from the imaging device and/or other sensors. For example, the controller 150 can recommend if a procedure is safe to proceed, e.g., based on characteristics of the tissue, nearby tissue structures, location of the focal point or focal volume relative to the target tissue, etc. Additionally, or alternatively, the controller 150 can determine whether an object and/or anatomy needs to be moved or avoided (e.g., a nerve or blood vessel). In some embodiments, the controller 150 can provide guidance on the type of tool or tool tip. For example, in some embodiments, the specified tool or tool tip can be a percutaneous tool or tool tip that can be used to move or avoid a nerve near the target area. In some embodiments, these percutaneous tools or instruments can be adjusted manually or controlled by the robotic system, e.g., to move to a target area, to move anatomical structures, etc. In some embodiments, the controller 150 can be situated nearby the other components of the system 100, such as, for example, a local computer, laptop, mobile device, tablet, etc. In some embodiments, the controller 150 can be remotely situated, such as a server or workstation that is remote from the other components of the histotripsy system 100. The controller 150 is further described with reference to FIG. 2A.

In some embodiments, the controller 150 can optionally be configured to communicate with third-party devices 160 via a network 102. The network 102 can include one or more network that may be any type of network (e.g., a local area network (LAN), a wide area network (WAN), a virtual network, a telecommunications network) implemented as a wired network and/or wireless network (e.g., Wi-Fi, Bluetooth®, Bluetooth® low energy, Zigbee, etc.) and used to operatively couple to any compute device (e.g., the controller 150). For example, the controller 150 can be configured to send information and/or receive information from one or more third-party devices 160.

The third-party devices 160 can include user devices (e.g., computer, mobile device, etc.), physician devices, databases, servers, etc. that are configured to communicate with the controller 150. In some embodiments, the third-party devices 160 can send instructions and/or commands to the controller 150. For example, the third-party devices 160 can send treatment plans and/or other information about a patient to the controller 150, which can cause the controller 150 to control one or more of the generator 130, robotic system 110, and/or histotripsy device 120 to follow a certain treatment path and/or to deliver HIFU with a predefined set of parameters (e.g., duty cycle, amplitude, intensity, etc.).

In some embodiments, the third-party devices 160 can receive information from the controller 150 or components coupled thereto. For example, the third-party device 160 can receive sensor measurements, positions and/or orientations of the robotic system 110 and the histotripsy device 120, and/or imaging data from the controller 150, the generator 130, the robotic system 110, the histotripsy device 120, and/or the imaging device 140. In some embodiments, the third-party devices 160 can be configured to receive and store the information for later reference, e.g., by a physician or other user. In some embodiments, the third-party devices 160 can be configured to generate, using information received from the controller 130, reports, visual representations, and/or other information for presentation to a user. In some embodiments, the third-party devices 160 can be configured to present such information to a user, e.g., via a user interface for communicating information associated with the operation of the histotripsy system 100. In some embodiments, the third-party devices 160 can display prompts for user input. The third-party devices 160 can then send the user inputs to the controller 150, which can be used to control one or more of the generator 130, robotic system 110, and/or histotripsy device 120.

In some embodiments, the system 100 can include one or more elements from systems and devices described in International Patent Application No. PCT/US2022/081891, titled “SYSTEM AND METHOD FOR TISSUE INTERVENTION VIA IMAGE-GUIDED BOILING HISTOTRIPSY,” filed Dec. 16, 2022, the disclosure of which is incorporated herein by reference.

FIG. 2A schematically depicts a controller 250 (e.g., structurally and/or functionally similar to the controller 150 of FIG. 1), according to embodiments. The controller 250 can be configured to process and/or analyze data (e.g., from other components of a HIFU system, such as, for example, system 100), present information, and/or generate commands and instructions.

The controller 250 can include a processor 252, a memory 254, an input/output device 256, and a communication interface 258 (or a multiplicity of such components). The memory 224 can be, for example, a random access memory (RAM), a memory buffer, a hard drive, a database, an erasable programmable read-only memory (EPROM), an electrically erasable read-only memory (EEPROM), a read-only memory (ROM), and/or so forth. In some embodiments, the memory 224 stores instructions that cause processor 222 to execute modules, processes, and/or functions associated with processing and/or analyzing data, presenting information, and generating commands and instructions for the other components of the histotripsy system. In some embodiments, the memory 224 can be configured to store information regarding patients, histotripsy procedures, etc.

The processor 252 can be any suitable processing device configured to run and/or execute functions associated with the operation of a HIFU system. These functions can include a transducer movement control 254a, a waveform parameter control 254b, an anatomical segmentation/boundary detection 254d, procedure monitoring 254e, and adaptive feedback and support 254f. In some embodiments, the processor 252 may be configured to execute only a subset of the aforementioned functions.

The processor 252 executing transducer movement control 254a can be configured to move a transducer array of a histotripsy device, such as the histotripsy device 120 of FIG. 1, according to a predetermined treatment path or trajectory, e.g., to mechanically fractionate a larger volume of tissue. The processor 252 can be configured to receive a predetermined treatment path as an input, e.g., from a user. For example, a physician may indicate a particular treatment path, e.g., via an input device, and the processor 252 can be configured to control the movement of the transducer array according to the treatment path. Alternatively, or additionally, the processor 252 may be configured to determine a treatment path (or portions thereof) based on information received about a patient (e.g., treatment volume, patient history, etc.).

When the treatment area is greater than a predetermined size, it may be desirable for the histotripsy device to mechanically fractionate the treatment area by following a treatment path. For example, the treatment path can include moving the focal point or focal volume of the histotripsy device to different locations according a predetermined pattern. In some embodiments, the treatment area may be a three-dimensional volume, and the treatment path may include delivering treatment (e.g., mechanically fractionating the tissue) in layers. In such embodiments, the treatment path can include delivering treatment from the deepest portion of the treatment area (e.g., the portion furthest from the transducer array or most distal) to the shallowest portion of the treatment area (e.g., the portion closest to the transducer array or most proximal). This path can reduce the effect of aberrations and/or changes in tissue structure (e.g., due to mechanical fractionation) in a more proximal area or section from affecting the HIFU waves as they target more distal areas or sections. It can also be desirable to control movement of the transducer array such that the focal point or focal volume of the HIFU waves does not induce undesirable heat build-up in one or more tissue portions. For example, the processor 252 can be configured to move the focal point or focal volume of the HIFU waves to apply boiling histotripsy to a plurality of tissue portions such that no one portion has an accumulated thermal dose or temperature that is greater than a predetermined threshold. In some embodiments, the processor 252 can be configured to move the focal point or focal volume of the HIFU waves according to a treatment path that enables previously treated portions to cool before receiving treatment again. This can help avoid undesirable thermal effects in the treated tissue portions. In some embodiments, the processor 252 can also be configured to pause the application of HIFU waves, e.g., to allow for heat induced by the HIFU waves to dissipate in one or more portions. In some embodiments, the processor 252 executing transducer movement control 254a can be configured to generate instructions and/or commands that, when sent to the robotic system (e.g., robotic system 110), cause the robotic system to move the histotripsy device to change the focus of the HIFU treatment according to the treatment path.

In some embodiments, the processor 252 executing transducer movement control 254a can be configured to determine or modify a treatment path for a target area, e.g., based on image data or other sensor data. For example, during a HIFU treatment, one or more imaging devices or sensors may monitor the progress of the HIFU treatment and/or other tissue characteristics, such as MRI data reflective of tissue contrast changes, temperature data, etc. Based on such information, the processor 252 may determine to continue moving a histotripsy device (e.g., a transducer array) according to a predetermined treatment path or to modify the treatment path. For example, the processor 252 may be configured to modify the treatment path such that the HIFU waves are directed at different tissue portions based on imaging or sensor data that reflects those tissue portions that have been mechanically fractionated and those that require additional HIFU application.

The processor 252 executing waveform parameter control 254b can be configured to control the parameters of the generated HIFU waves. For example, the processor 252 can be configured to control a generator (e.g., generator 130) to generate pulse waveforms according to set parameters, which in turn can drive a transducer array to produce ultrasound waves having desirable properties. The various parameters can include, for example, power, power density, intensity, oscillation frequency, pulse duration, PRF, duty cycle, and a number of pulses. In some embodiments, the processor 252 can be configured to receive waveform parameters from a user, e.g., via an input device. Alternatively, or additionally, the processor 252 can be configured to determine and/or modify one or more waveform parameters based on information received regarding a patient (e.g., treatment volume, patient history) and/or imaging or sensor data collected regarding a treatment area. In some embodiments, modifying the output can include modifying the power, power density, intensity, oscillation frequency, pulse duration, PRF, duty cycle, and number of pulses. In some embodiments, the processor 252 can be configured to determine the waveform parameters based on the treatment path or trajectory indicated for treating a tissue volume, and vice versa. For example, when the treatment path for applying the HIFU waves involves treating across multiple tissue portions of a larger region or volume, the processor 252 can be configured to control the transducer array to move its focal point or focal volume across the multiple portions according to a predefined protocol (e.g., at a predefined rate) while controlling the generator to deliver ultrasound pulses at an increased duty cycle and/or PRF. Such can enable faster treatment time for the entire volume, while avoiding thermal build-up in any one tissue portion that exceeds an upper threshold. In other words, for any one tissue portion, the processor 252 can be configured to control the delivery of HIFU waves to deliver doses of HIFU energy with sufficient time separation between doses to allow induced heat to dissipate and remain within desirable ranges.

The processor 252 executing anatomical segmentation/boundary detection 254d can be configured to generate a visual representation of the target area and/or the components of the histotripsy system based on received imaging data. The imaging data can include computerized tomography (CT) data, ultrasound imaging data, magnetic resonance imaging (MRI) data, and/or the like. The processor 252 can be configured to receive pre-operative and/or intra-operative imaging data of a patient's anatomy, which it can use to perform anatomical segmentation of regions of interest. In some embodiments, the processor 252 can be configured to receive a pre-operative image dataset that is captured using a higher resolution modality, e.g., a CT or MRI scan. Then subsequently, during a treatment procedure, the processor 252 can be configured to receive an intra-operative imaging dataset, e.g., captured using a lower resolution imaging modality such as ultrasound, and to perform segmentation, object or boundary detection, and/or the like to register the two image datasets to one another. In particular, the processor 252 can be configured to perform segmentation on the two image datasets to identify features that exist across the two image dataset (e.g., anatomical features). In some embodiments, the output of the segmentation can include a point cloud of the anatomical features. The processor 252 can be configured to register the first image dataset and/or the point cloud associated therewith with the second image dataset and/or point cloud associated therewith in a common coordinate system. Registration in a common coordinate system allows for the anatomy of the patient and for the ultrasound imaging device to have known relative positions. In some embodiments, the ultrasound imaging device may be fixedly coupled to a histotripsy device such that the position of the histotripsy device may also be registered in common coordinate system. In some embodiments, the processor 252 can be configured to generate visual representations of the patient's anatomy and/or one or more components of the HIFU system with greater fidelity or detail, based on the registration.

Optionally, the processor 252 executing procedure monitoring 254e can be configured to monitor the procedure via an imaging device (e.g., camera, etc.) and/or other sensors. For example, the processor 252 can receive tracking data associated with at least one component of the histotripsy system (e.g., system 100). The position tracking data can include the position of the robotic system, the histotripsy device, a surgical device, an ultrasound imaging device, and/or the like. The tracking data can be captured using the imaging device, such as a camera configured to capture infrared, optical, ultraviolet, or other markers. The processor 252 can register the position tracking data with the common coordinate system. Registering the position tracking data with the common coordinate system can allow the processor 252 to monitor the operation of the histotripsy system, e.g., to confirm that the system is operating according to expected protocols. In some embodiments, the processor 252 can also generate a visual representation of the target area, including obstacles (e.g., bones, nerves), and the progress of the histotripsy treatment. The visual representation can be used to assist an operator in operating the histotripsy system or for observing the histotripsy system to ensure the histotripsy system is operating as desired.

The processor 252 executing adaptive feedback and support 254f can be configured to provide a user or an operator with feedback regarding the status of the target area and/or the operation of the histotripsy system. For example, the processor 252 can determine the volume of liquified tissue around a nerve after boiling histotripsy has been delivered. Based on the volume of liquified tissue, and/or patient anatomy, the adaptive feedback and support 254f can provide the user with a recommendation on the type of tool that is desired for moving the nerve. As another example, the processor 252 can determine a treatment plan based on an analysis of a portion of the tissue in the target area (e.g., collected using a biopsy device). For example, the processor 252 can generate a recommendation based on the analysis. The processor 252 may be configured to receive user feedback to adjust the generated recommendation and/or the implementation of the recommendation.

As noted above, the controller 250 can include one or more input/output device(s) 256. The input/output device 256 of the controller 250 can include a display, audio device, or other output device for presenting information to a user. For example, the output device can be configured to present (e.g., via a user interface) the subject's data and/or reports or output data associated with the procedure. Additionally, or alternatively, the output device can be configured to present (e.g., via a user interface) information showing recommendation and/or prompts associated with the procedure. In some embodiments, the input/output device 256 can include or be operatively coupled to a touchscreen, a keyboard, or other input device for receiving information from a user. The input/output device 256 can be configured to integrate one or more of user instructions, modifications to operation of the histotripsy device, treatment plan, and/or the like. The input/output device 256 can allow for the user to select an option on the controller, the option, for example, indicating if a procedure should continue, be modified, and/or be paused or terminated. In some embodiments, the input/output device 256 can display a visualization of at least a portion of the procedure, procedure progress, and/or the like.

The communications interface 258 of the compute device 250 can be configured to receive information and/or send information to other components of a histotripsy system. The communications interface 258 can be a wired or wireless communications interface. As described with reference to FIG. 1, the controller 250 can be configured to communicate via a network with one or more third-party devices. This can be facilitated via the communications interface 258.

FIG. 2B schematically depicts a histotripsy device 210 (e.g., functionally and/or structurally similar to the histotripsy device 110 of FIG. 1), according to embodiments. As schematically illustrated, the histotripsy device 210 can be configured to engage with a patient P. The histotripsy device 220 includes, optionally, sensor(s) 222, optionally, an imaging transducer 224, an actuator 225, a treatment transducer array 226, and an interface 228. In some embodiments, the histotripsy device 210 may be coupled to a robotic system (e.g., structurally and/or functionally similar to the robotic system 110 of FIG. 1) and/or a generator (e.g., structurally and/or functionally similar to the generator 130 of FIG. 1).

The sensor(s) 222 may include one or more sensors configured to measure a characteristic of at least one of the histotripsy device 220 or the patient P. For example, the sensor(s) 222 can include at least one temperature sensor for determining the temperature of a portion of the patient P's anatomy and/or a temperature sensor for determining the temperature of the transducer array 226. The sensor(s) 222 can include a position and/or an orientation sensor that can measure the movement of the histotripsy device 220 and/or determine its focal point or focal volume. In some embodiments, the sensor(s) 222 can include sensors that can measure the output of the histotripsy device 202 (e.g., of the transducer array 226) and/or the input energy received by the histotripsy device 220 from the generator. In some embodiments, the sensor(s) 222 can include receiving elements, e.g., one or more transducers that can be configured to measure HIFU energy reflected by a patient. The measurements can provide information regarding a timing of receiving and/or intensity of the reflected energy. Including such sensors allows for the output of one or more treatment transducers to be monitored so that the output can be adjusted to provide desired (e.g., desired location, desired power, etc.) HIFU at the focal point or focal volume. For example, based on the signals from the receiving elements, the intensity, firing sequence, and/or position of the treatment transducers may be adjusted, thus allowing for adjustment of the treatment transducers during operation. This further allows for the transducer array 226 output to be adjusted during motion of the transducer array 226. Further details of such sensors are described in International Patent Application No. PCT/IB2025/051100, titled “SYSTEMS, DEVICES, AND METHODS FOR NON-INVASIVE TREATMENT OF TISSUE USING BOILING HISTOTRIPSY,” filed Jan. 31, 2025, the disclosure of which is incorporated herein by reference in its entirety.

The imaging transducer 224 may be configured to allow for a user to monitor the treatment area while the histotripsy device 220 is in operation. In some embodiments, the imaging transducer 224 is an ultrasound imaging transducer. The imaging transducer 224 may be generally concentric with the transducer array 226 or may be located offset from the transducer array 226. In some embodiments, the histotripsy device 220 may include more than one imaging transducer 224 to capture multiple views of the treatment area. For example, one imaging transducer 224 may be concentric with the transducer array 226 and another imaging transducer 2245 may provide a perspective view of the target area.

The actuator 225 is configured to receive an input from a user and/or a controller (e.g., structurally and/or functionally similar to the controller 150 of FIG. 1 and/or the controller 250 of FIG. 2). Based on the input, the actuator 225 may be configured to activate at least one function of the histotripsy device 220. For example, when the actuator 225 is activated, the histotripsy device 220 may deliver treatment to the patient P. In some embodiments, the actuator 225 is a switch, a button, and/or the like.

The treatment transducer array 226 is an array of transducers configured to deliver HIFU to the patient P. In some embodiments, the transducer array 226 is configured to induce boiling histotripsy at a target area. The transducer array 226 may include a plurality of transducers, each configured to deliver a portion of a total HIFU output. The transducer array 226 is configured such that the ultrasonic waves from the transducers are focused on a focal point or focal volume to create a pulsatile wavefront of ultrasound radiation directed at the focal point or focal volume. For example, the transducers of the transducer array 226 may be arranged so that waves generated by each transducer converge at the focal point or focal volume. This allows the transducer array 226 to be focused on a target area, or a portion of the target area. In some embodiments, the transducers of the transducer array 226 can be arranged in a ring or circle, e.g., with the transducers disposed around the ring. In some embodiments, the transducer array 226 can be arranged in an arch. The shape of the transducer array 226 may be configured for a specific usage. In some embodiments, the transducer array 226 may be configured to allow for certain transducers to be selectively turned off to alter the output of the histotripsy device 220. In some embodiments, the transducer array 226 may include articulating transducer elements that allow for the focal point or focal volume of the transducer array 226 to be moved.

The interface 228 is configured to provide an interface between the transducer array 226 and the patient P that allows HIFU waves to travel into the patient P without much distortion, so that HIFU can effectively be applied to the patient P. The interface 228 can be a tank, bladder, bag, and/or the like of an interfacing fluid. In some embodiments, the fluid is deionized water, degassed water, and/or the like. In some embodiments, the interface 228 includes a gasket that is configured to form a seal with the patient P. In some embodiments, a partial vacuum can be drawn in the interface to allow for a seal between the interface and the patient P. In some embodiments, the volume of fluid within the interface 228 (e.g., fluid within a bladder) can be increased or decreased (e.g., via removal or leakage, or refilling) for allowing adjustments to the position and/or orientation of the transducer array 226. For example, fluid can be added to a bladder to increase a distance between the transducer array 226 and the patient P, or fluid can be removed from the bladder to decrease a distance between the transducer array 226 and the patient P. Fluid can also be removed (or allowed to leak out) as the transducer array 226 may be moved (e.g., by a robotic system) to change its focal point or focal volume. In some embodiments, the fluid within the interface 228 can be temperature controlled (e.g., cooling and/or heating) to regulate the temperature of the fluid or maintain the temperature of the fluid at a set temperature. For example, a temperature sensor can be used to monitor the temperature of the transducers and/or cooling fluid, and when such temperatures reach certain thresholds, the histotripsy system may be configured to pause operation (e.g., stop delivery of HIFU pulses to the transducer array) and/or implement additional active cooling (e.g., by replacing the cooling fluid with new fluid, by activating a fan or other active cooling mechanism, by delivering additional fluid to other sides of the transducer, etc.). In some embodiments, the interface 228 can be configured to slide or move along the skin of the patient P, e.g., to enable movement of the focal point or focal volume of the transducer array 226. For example, the interface 228 can be made of a low friction material that can slide along the body of the patient P. Alternatively, or additionally, the interface 228 can be covered with a fluid (e.g., fluid that has leaked out of the bladder) or lubricant that allows for the interface 228 to slide/move along the surface of the patient P. In some embodiments, the interface 228 is flexible to allow for movement of the histotripsy device to conform to the patient P.

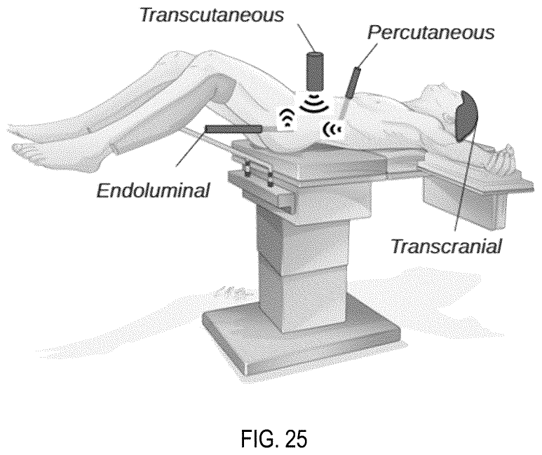

While the histotripsy device 220 (and other histotripsy devices described herein) are described as extracorporeal devices (e.g. transcutaneous), it can be appreciated that the histotripsy device 220 can also be an endoluminal device, percutaneous device, and/or transcranial device as depicted in FIG. 25.

FIG. 2C schematically depicts a robotic system (e.g., structurally and/or functionally similar to the robotic system 110 of FIG. 1, according to embodiments. The robotic system 210 includes a processor 212, a memory 214, and a robotic manipulator 216. The robotic system 210 can be a component of the histotripsy system, for example, the histotripsy system 100 of FIG. 1. In some embodiments, the robotic system 210 is coupled to at least one of a histotripsy device (e.g., structurally and/or functionally similar to the histotripsy device 120 of FIG. 1 and/or the histotripsy device 220 of FIG. 2B) and/or an imaging device (e.g., structurally and/or functionally similar to the imaging device 140 of FIG. 1). The robotic system 210 can be configured to reposition and/or reorient the histotripsy device and/or the imaging device to engage the treatment area.

The memory 214 can be, for example, a random access memory (RAM), a memory buffer, a hard drive, a database, an erasable programmable read-only memory (EPROM), an electrically erasable read-only memory (EEPROM), a read-only memory (ROM), and/or so forth. In some embodiments, the memory 214 stores instructions that cause processor 212 to execute modules, processes, and/or functions associated with operation of the robotic system 210. In some embodiments, the memory 224 stores information associated with a treatment path and/or a histotripsy device, such as the histotripsy device 120 of FIG. 1 and/or the histotripsy device 220 of FIG. 2B. In some embodiments, memory 224 can store information in a user profile. For example, the user profile can include information regarding multiple procedures associated with a user. In some embodiments, the memory 224 can store information regarding the position and/or orientation of the robotic system 210.

The processor 252 can be any suitable processing device configured to run and/or execute functions associated with the operation of a robotic system 210. The processor 252 can be configured to execute instructions related to moving the histotripsy device (or treatment transducer array of the histotripsy device) based on a treatment path to apply treatment to a treatment area. For example, the instructions can include instructions for how and when the robotic manipulator 216 should be manipulated based on the treatment plan. In some embodiments, the robotic system 210 may receive instructions. In some embodiments, the robotic system 210 may receive a treatment path and the processor 212 can generate instructions for operating the robotic manipulator 216 according to the treatment path.

Referring generally to the macro positioning device 216 and the micro positioning device 218, the robotic system 210 is configured to reposition and/or reorient the histotripsy device and/or the imaging device coupled to the robotic system 210. The macro positioning device 216 is configured to position the histotripsy device so that the histotripsy device engages the patient. In some embodiments, the macro positioning device 216 is a robotic arm, for example, a robotic arm including a plurality of joints and segments. In some embodiments, the macro positioning device 216 is a robotic arm formed of one or more segments, each coupled at a joint that couples one or more segments to one another and allows for movement of one or more segments in at least one degree of motion (e.g., a translational and/or rotational degree of freedom). In some embodiments, the joints and/or segments can be configured to translate, rotate, and/or the like.

The micro positioning device 218 is coupled to a distal end of the macro positioning device 216. The histotripsy device (including one or more treatment transducers and/or imaging transducers) can be coupled to the micro positioning device 218. For example, the transducer array and/or the imaging system can be coupled to the distal end of the micro positioning device 218. The micro positioning device 218 is configured to position the histotripsy device and, thus, control the location of the focal volume. The micro positioning device 218 can be configured to position an imaging transducer to capture image data of an anatomical region of a patient. In some embodiments, the micro positioning device 218 can include one or more joints each configured to allow for at least one degree of motion (e.g., a translational and/or rotational degree of freedom). In some embodiments, the joints can include any number and/or combination of revolute (e.g., rotatory) and/or prismatic (e.g., linear) joints. The micro positioning device 218 is configured to move the focal volume along the target area while delivering treatment. Additionally, the micro positioning device 218 can be configured to move the imaging transducer during treatment so that the imagine transducer can desirably image the target area during treatment.

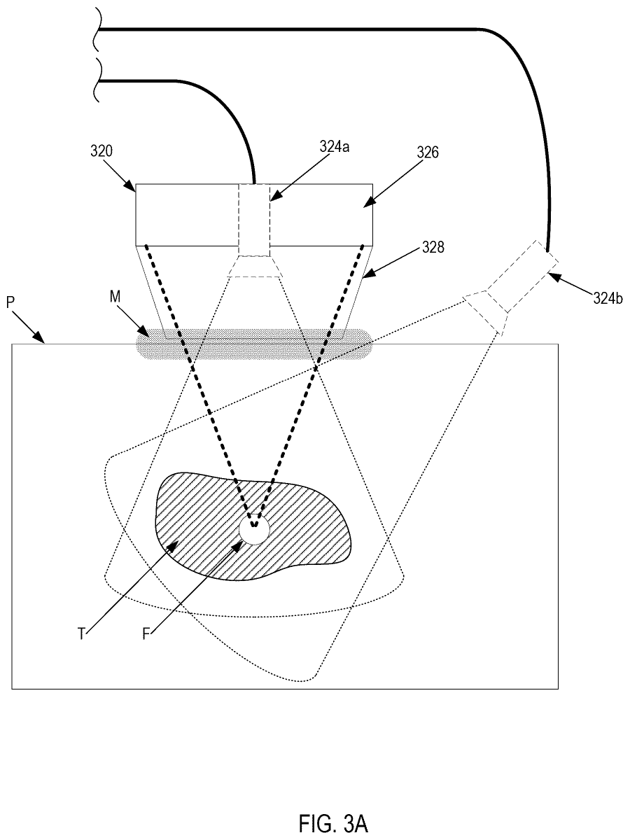

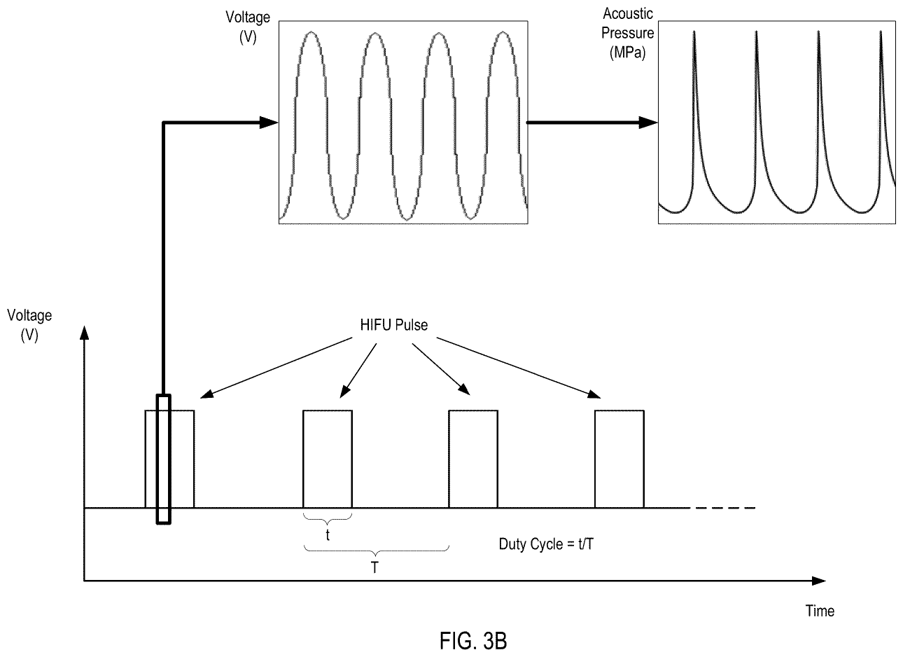

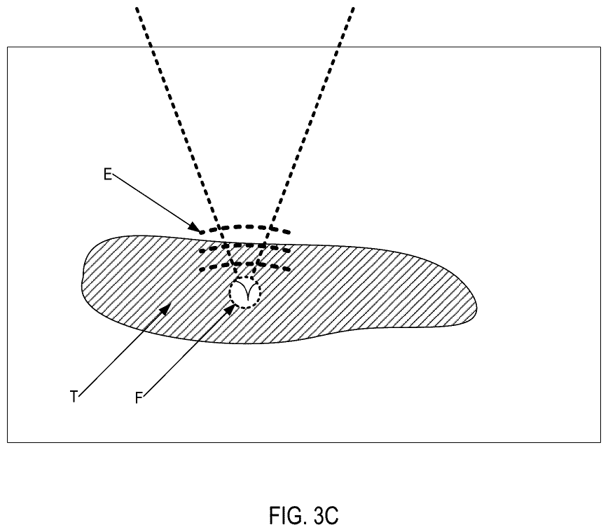

FIG. 3A depicts a histotripsy device implemented as a transducer device 320 (e.g., functionally and/or structurally similar to the histotripsy device 120 of FIG. 1 and/or the histotripsy device 220 of FIG. 2B) operatively engaging tissue T of the patient P, according to embodiments. The transducer device 320 includes one or more imaging transducers 324a and 324b (e.g., structurally and/or functionally similar to the imaging transducer 224 of FIG. 2B), a transducer array 326 (e.g., functionally and/or structurally similar to the transducer array 225 of FIG. 2B), and an interface 328 (e.g., functionally and/or structurally similar to the interface 228 of FIG. 2B). The imaging transducers 324a and 324b are shown in dashed lined to indicate that each can be optional. For example, the device 320 can include a single imaging transducer 324a, or a single imaging transducer 324b, or neither of the imaging transducers 324a or 324b. In the latter case, a separate imaging device (not shown in FIG. 3A) may be used with the transducer device 320, e.g., to capture intraoperative images of the patient P. In embodiments, the interface 328 of the transducer device 320 can be engaged with the patient P, and a medium M can be disposed between the interface 328 and the patient P. The engagement medium M can be an ultrasound gel or a similar substance that reduces friction between the transducer device 320 and the patient P.

The transducer array 326 of the transducer device 320 can be configured to generate lesions in tissue at one or more focal points (or focal volumes) F. The focal point F may be the precise point or volume where waves produced by the transducer array 326 converge. During operation of the transducer device 320, the transducer device 320 can be positioned and/or oriented relative to the patient body such that the focal point F is located within a target tissue area T. Further, during operation, the focal point F can be moved (e.g., via movement of the transducer device 320 and/or the patient P) according to a treatment path to deliver energy to multiple portions within the treatment area of the tissue T, e.g., so that treatment can be delivered to the entire treatment area.

The imaging transducer 324a is optionally concentrically located with the transducer array 326. The imaging transducer 324a can provide a top-down view of the tissue T during operation of the transducer device 320. The imaging transducer 324b is optionally located separate from the transducer device 320 to generate a different view (e.g., a side view) of the tissue T. In some embodiments, the transducer device 320 includes neither, one of, or both the transducer 324a and the transducer 324b.

The interface 328 can be configured to engage with a tissue surface of the patient P, so that treatment can be effectively applied to the tissue T. The interface 326 can include a medium (e.g., fluid, gel, gas, etc.) that allows for the ultrasonic waves from the transducer array 326 to travel to the tissue T without an undesirable (e.g., less than a threshold) amount of energy lost and/or distortions in the waves. The interface 328a can be configured to be sealed to the patient P (e.g., via suction and/or a seal/gasket) or may be moveable along the patient P. In some embodiments, the interface 328 is configured to selectively seal to the patient P. For example, the interface 328 may be configured to seal to a first position on the patient P, then be removed, then be resealed on a different position of the patient P. In some embodiments, the interface 328 can be flexible to allow for the transducer device 320 to be reoriented or repositioned, e.g., by deforming the interface 328. In some embodiments, the size of the interface 328 can also be changed, e.g., by filling and/or removing fluid from the interface. In an embodiment, the interface is a bladder or other fluid reservoir that can be filled with a fluid, such as, for example, a degassed fluid.

FIG. 3B depicts a graph showing a HIFU voltage pulse waveform, which can be used to drive a transducer array to generate HIFU waves, according to embodiments. The graph shows HIFU pulses, which occur for a time period t. The duty cycle of HIFU treatment is defined as the time period t divided by a total time T, which may be the time from the beginning of a pulse to the beginning of a second pulse. The frequency of the HIFU pulses defines the PRF. Each pulse is composed of a HIFU wave that oscillates at an oscillation frequency of greater than 1 MHz and less than 3 MHz, including all sub-ranges and values therebetween. In some embodiments, a transducer array (e.g., of a histotripsy device), in response to receiving the pulse waveform, can generate ultrasonic waves having a peak negative acoustic pressure of greater than about −35 MPa (e.g., between about −25 MPa and about −20 MPa, or between about −35 MPa and about −25 MPa, and/or other ranges greater than −35 MPa) to induce cavitation cloud histotripsy. In some embodiments, a transducer array (e.g., of a histotripsy device), in response to receiving the pulse waveform, can generate ultrasonic waves having a peak negative acoustic pressure of greater than about −15 MPa (e.g., between about −15 MPa and about −10 MPa) and a peak positive acoustic pressure of greater than about 70 MPa (e.g., between about 70 MPa and about 120 MPa, and/or other ranges greater than −70 MPa) to induce boiling histotripsy or acoustic jet liquefaction. The voltage, the frequency, the duty cycle, and/or the PRF can be modified to alter the output of the transducer. In some embodiments, boiling histotripsy or acoustic jet liquefaction can be configured to treat stiffer tissue and can have a larger focal length or F (hence a smaller diameter for the same depth), which can enable small treatment heads (e.g., on probes and/or catheters) to access more spaces. For example, smaller treatment heads can be configured to access more spaces in a transcutaneous manner (e.g., thyroid, breast, prostate via peritoneum), or deeper/stiffer tissues (pancreas). In some embodiments, a smaller treatment head can be placed on a smaller end effector that can be positioned at the end of a probe or flexible catheter to access target regions from the inside out.

FIG. 3C schematically depicts ultrasound energy E being delivered to tissue, according to embodiments. The ultrasound energy E may be produced by a histotripsy device (or a transducer array thereof), such as any of the histotripsy devices described herein. The ultrasound energy E can be delivered to tissue T of a patient at a focal point (or focal volume) F. With boiling histotripsy or acoustic jet liquefaction, at the focal point F, the ultrasound energy E heats the tissue such that the tissue forms a bubble. Further interactions between positive pressure waves (e.g., shock waves) and the bubble can cause mechanical fractionation and/or cavitation. The positive pressure waves can induce a whirlpool effect with one or more acoustic jets that form an acoustic fountain that extends from the initial bubble. The tissue T at the focal point F is atomized, emulsified, and/or destroyed by the fractionation and/or cavitation of the bubble. The ultrasound energy can be delivered in a manner that reduces non-linear heating affects, such that the effects on the tissue are predominantly mechanical.

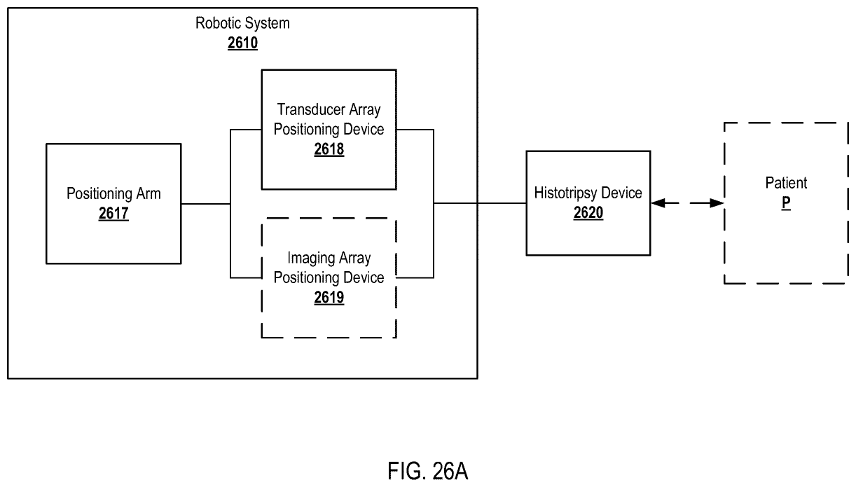

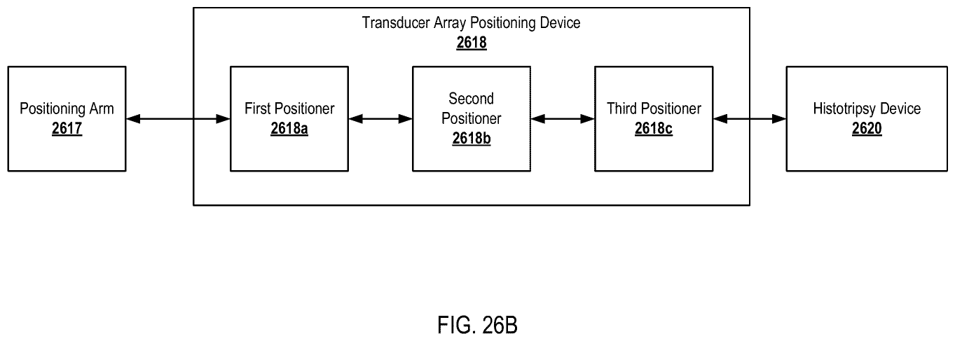

In some embodiments, the robotic systems described herein can include a first robotic assembly (such as, for example, a robotic arm) that can provide macro or gross positioning of one or more components of a histotripsy device and a second robotic assembly (such as, for example, a delta robot assembly or a cartesian robot assembly) that can provide micro or finer positioning of one or more components of a histotripsy device. For example, FIGS. 26A-26B schematically depict a robotic system 2610 (e.g., functionally and/or structurally similar to the robotic system 110 of FIG. 1 and/or the robotic system 210 of FIG. 2C) positioning a histotripsy device 2620 (e.g., functionally and/or structurally similar to the histotripsy device 120 of FIG. 1, the histotripsy device 220 of FIG. 2B, and/or the histotripsy device 320 of FIG. 3A) of a HIFU system, according to embodiments. As seen in FIG. 26A, the robotic system 2610 includes a positioning arm 2617 (e.g., e.g., functionally and/or structurally similar to the macro positioning device 216 of FIG. 2C) and a transducer array positioning device 2618 and, optionally, an imaging array positioning device 2619 (the transducer array positioning device 2618 and the imaging array positioning device 2619 being part of a micro positioning device, e.g., functionally and/or structurally similar to the micro positioning device 218 of FIG. 2C).

The positioning arm 2617 is coupled to the transducer array positioning device 2618 and/or the imaging array positioning device 2619 (collectively referred to as the “positioning devices 2618, 2619”). The transducer array positioning device 2618 and/or the imaging array positioning device 2619 are coupled to the histotripsy device 2620. The positioning arm 2617 is configured to position the positioning devices 2618, 2619 and, thus, the histotripsy device 2620, at a location that is proximate to the patient P or within an anatomical region of the patient P. The positioning arm 2617 may be configured to operate between a disengaged (e.g., storage, transport, etc.) configuration, in which the histotripsy device 2620 is positioned away from the patient P, and an engaged configuration where the histotripsy device 2620 engages the patient P (e.g., via an interface). In some embodiments, the positioning arm 2617 may be a robotic arm with at least three degrees of freedom (DOFs) that is configured to position and orient the histotripsy device 2620 in a desired location (e.g., adjacent to the treatment area) on the patient P. For example, the positioning arm 2617 can be configured to position the histotripsy device 2620 and orient the history device 2620 so that treatment can be delivered to a target area for treatment and such that a focal volume of the histotripsy device 2620 is adjacent to the target area.

The transducer array positioning device 2618 is configured to position or move a transducer array (e.g., functionally and/or structurally similar to the treatment transducer array 226 of FIG. 2B and/or the transducer array 326 of FIG. 3A) such that the focal volume can be moved throughout the target area, e.g., to induce boiling histotripsy at multiple focal volumes that collectively enable treatment of the entire target area. In some embodiments, the transducer array positioning device 2618 is configured to move the histotripsy device 2620 in at least three DOFs to cause the focal volume to move through a three-dimensional target area. In some embodiments, the transducer array positioning device 2618 can be configured to rotate the transducer array.

As seen in FIG. 26B, the transducer array positioning device 2618 can include a plurality of positioners including a first positioner 2618a, a second positioner 2618b, and a third positioner 2618c. The positioners 2618a, 2618b, 2618c can include one or more joints that enable movement of the transducer array in one DOF. For example, the first positioner 2618a is configured to allow the transducer to move along a first DOF. In some embodiments, the first positioner 2618a can be a revolute joint (e.g., rotary joint, rotary actuator, etc.) or a prismatic joint (e.g., linear actuator, prismatic actuator, etc.). The second positioner 2618b is configured to allow the transducer to move along a second DOF, different from the first DOF. In some embodiments, the second positioner 2618b can be a revolute joint (e.g., rotary joint, rotary actuator, etc.) or a prismatic joint (e.g., linear actuator, prismatic actuator, etc.). The third positioner 2618c is configured to allow the transducer to move along a third DOF, different from the second DOF and the first DOF. In some embodiments, the third positioner 2618c can be a revolute joint (e.g., rotary joint, rotary actuator, etc.) or a prismatic joint (e.g., linear actuator, prismatic actuator, etc.). The positioners allow for the focal volume of the histotripsy device 2620 to be moved throughout a three-dimensional target area to treat the three-dimensional target area using boiling histotripsy. In some embodiments, the positioners can include motors, actuators (e.g., rotary actuator, prismatic actuator, etc.), and/or the like to move the histotripsy device 2620. In some embodiments, the positioners can include arms of a delta robot assembly. In some embodiments, such as when the positioners are in a delta robot assembly, the positioners can include a rotary motor that is configured to rotate an input of a rotary-to-linear transmission to cause translation of a proximal end of the respective positioner and, thus, of the histotripsy device 2620. In some embodiments, the first positioner 2618a, the second positioner 2618b, and the third positioner 2618c can be any combination of revolute joints and/or prismatic joints. For example, all can be prismatic joints, such as when the positioners form part of a cartesian robot assembly. In some embodiments, the transducer array positioning device 2618 can include any number of additional positioners, e.g., one more, two more, or three more additional positioners or joints. For example, the transducer array positioning device 2618 can include additional positioners for rotating, translating, and/or orienting the transducer array.

As shown in FIG. 26A, the imaging array positioning device 2619 is configured to position an imaging transducer (e.g., functionally and/or structurally similar to the imaging transducer 224 of FIG. 2B) of the histotripsy device 2620 so that the imaging transducer operatively engages the patient P. For example, the imaging array positioning device 2619 can be configured to translate and/or rotate the imaging array positioning device 2619 so that it engages the patient P to produce a desired image of the target area. In some embodiments, the positioning devices 2618, 2619 are at least partially disposed within an interface (e.g., functionally and/or structurally similar to the interface 228 of FIG. 2B). For example, the positioning devices 2618, 2619 may be configured to operate within a fluid inside the interface.

Further details of a treatment transducer positioning device and an imaging transducer positioning device are provided in International Patent Application No. PCT/US2025/025214, titled “HISTOTRIPSY SYSTEMS INCLUDING STEERABLE ARRAYS, AND DEVICES AND METHODS THEREOF,” filed Apr. 17, 2025, the disclosure of which is incorporated herein by reference.

### II. Positioning Arm Designs

As described above, systems, devices, and methods described herein can include robotic systems, which can be configured to manipulate, control, operate, etc. one or more components of a histotripsy device, such as, for example a BH device.

Histotripsy systems as described herein can include a distal end having a an end effector assembly, which can also be referred to as a distal robotic assembly and/or a therapeutic delivery robot. The end effector assembly can include one or more positioning devices (e.g., structurally and/or functionally similar to transducer array positioning device 2618 and/or imaging array positioning device 2619) that can be configured to support and/or manipulate a histotripsy device (e.g., histotripsy device 120, 220, and/or any other histotripsy device described herein). As described above, the histotripsy systems can be configured to deliver BH to tissue, e.g., to treat cancerous tissue and/or ablate other tissue structures.

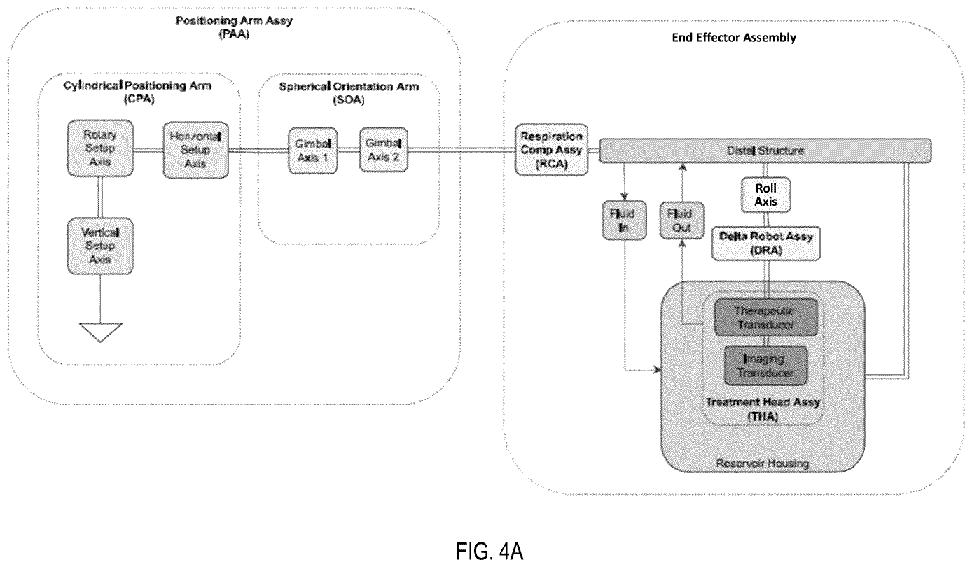

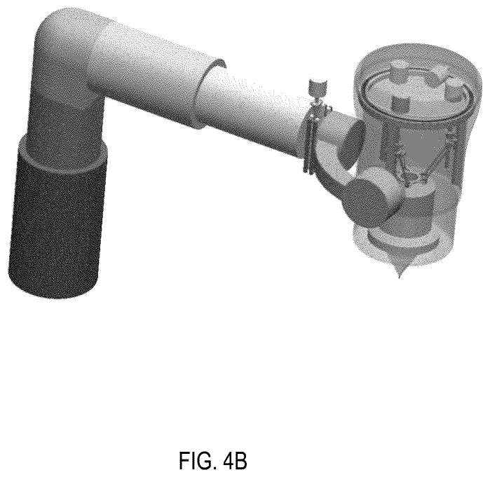

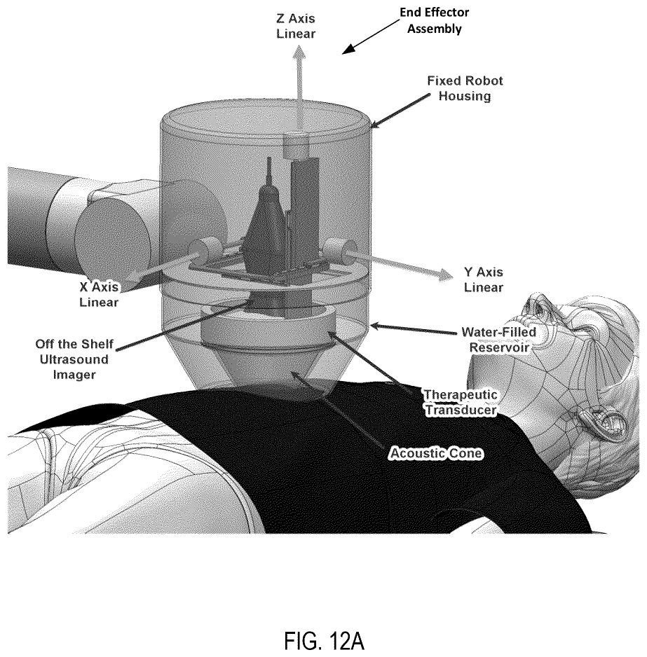

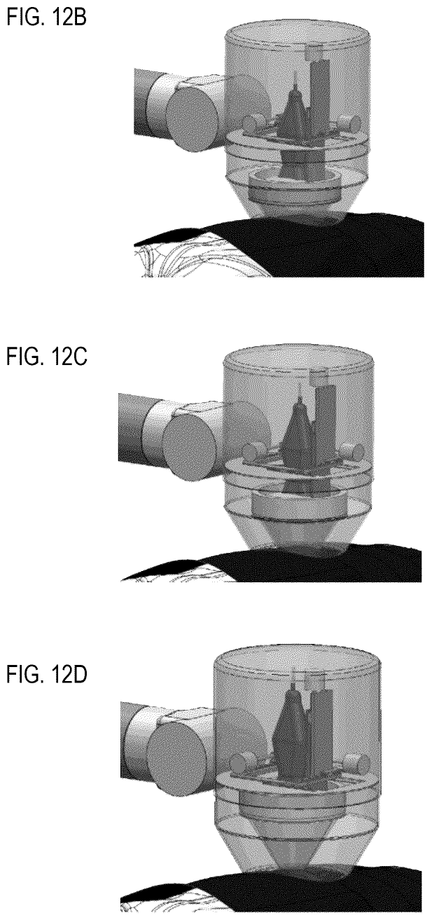

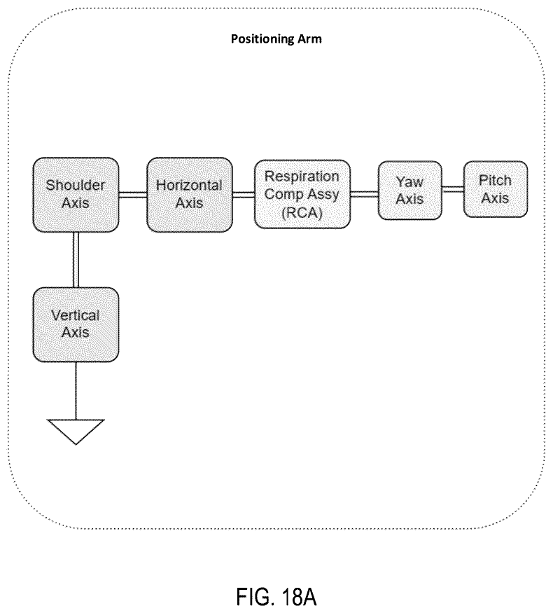

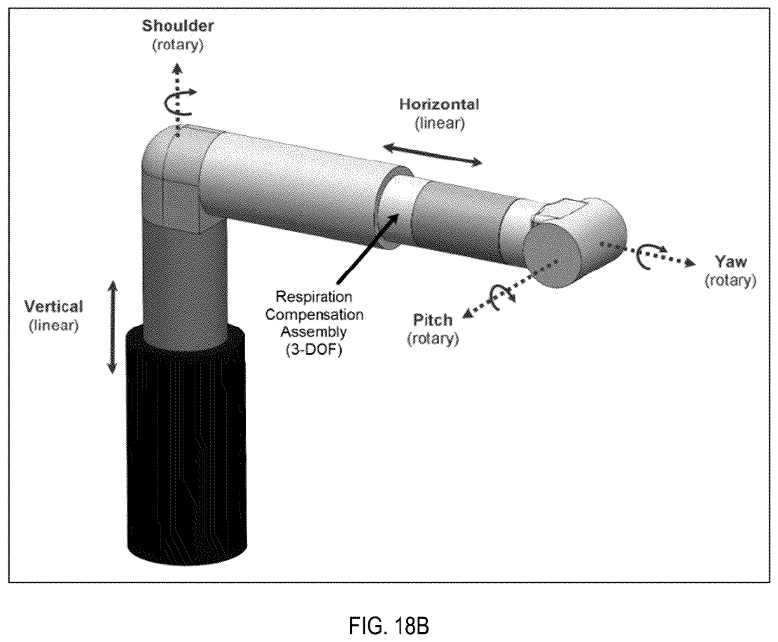

FIGS. 4A and 4B depict a first example of a HIFU system, including a positioning arm assembly and an end effector assembly, according to embodiments. FIG. 4A schematically depicts the components of the HIFU system, and FIG. 4B depicts the positioning arm assembly and the end effector assembly. The HIFU system depicted in FIGS. 4A and 4B can be structurally and/or functionally similar to other histotripsy or HIFU systems described herein. The positioning arm assembly can include a cylindrical positioning arm configured to provide movement along or about a vertical setup axis (e.g., to allow for vertical height adjustment, e.g., as shown in FIG. 18B), a rotary setup axis (e.g., to allow for rotation about a vertical or a horizontal axis of the arm), and a horizontal setup axis (e.g., to allow for horizonal adjustment, e.g., as shown in FIG. 18B). The positioning arm assembly can also include a spherical orientation arm, e.g., having one or more gimbals, which can be configured to provide rotation about one or more axes. As shown, two gimbals can be used to provide rotation about two gimbal axes, gimbal axis 1 and gimbal axis 2 (e.g., yaw and pitch axes). The end effector assembly can include a respiration compensation assembly, further described with reference to FIGS. 12A-12D, and a distal structure that supports a treatment head assembly and a reservoir housing. The treatment head assembly can be configured to deliver HIFU, such as, for example, BH, to treat tissue. The treatment head assembly can be supported by a treatment transducer positioning device (e.g., a delta robot assembly), which can be configured to enable movement of the treatment head in at least one DOF (e.g., to rotate about one or more axes and/or translate about one or more axes). For example, the treatment transducer positioning device can be configured to enable movement of the treatment head about at least 3 DOFs. In some embodiments, the end effector assembly can also include an imaging transducer positioning device, which can be configured to move an imaging device (e.g., any of the imaging transducers described herein). While FIGS. 4A and 4B implement the treatment transducer positioning device as a delta robot assembly, it can be appreciated that end effector assemblies can include any type of treatment transducer positioning device, including a delta robot, cartesian robot, and/or other treatment transducer positioning devices.

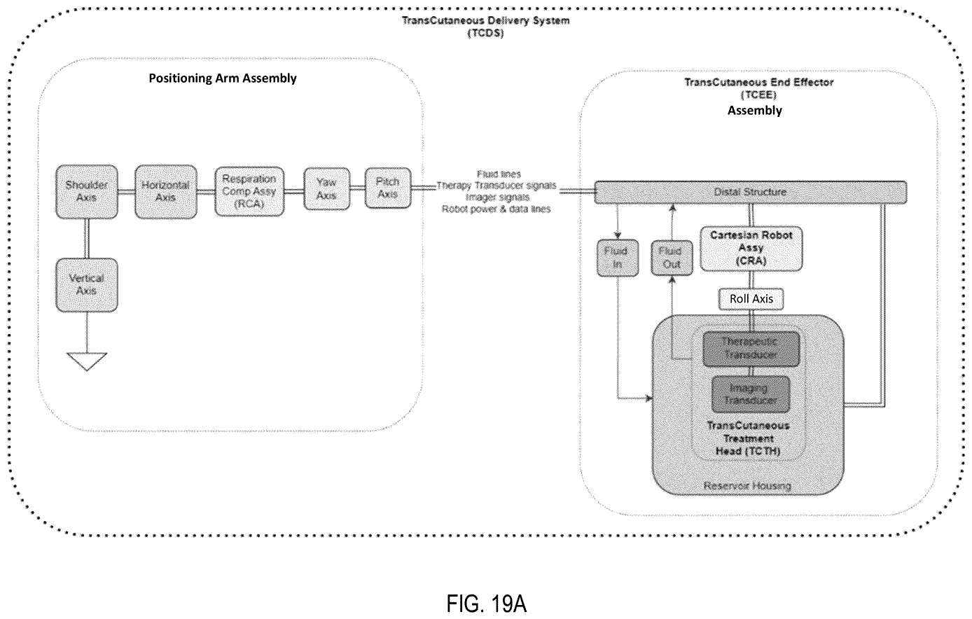

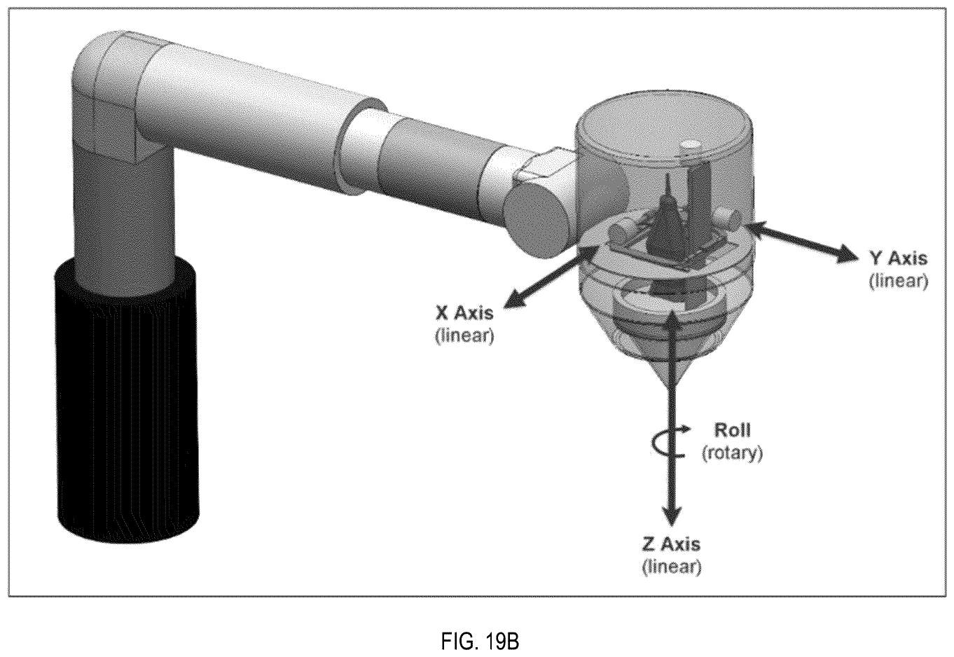

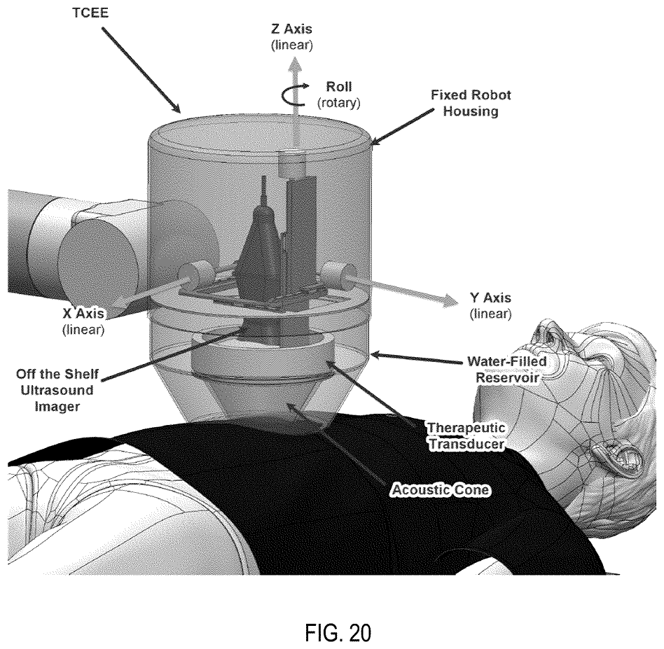

Optionally, a joint or actuator can be coupled to the treatment head assembly to provide motion about a roll axis (e.g., a longitudinal axis of the treatment head assembly, or an axis that extends between proximal and distal ends of the treatment head assembly, e.g., as shown in FIGS. 19B and 20). The reservoir housing can be configured to hold a fluid, e.g., for providing an interface (e.g., interface 228) for treating the patient. The end effector assembly can include fluid channels for supplying fluid to and receiving fluid from the reservoir housing.

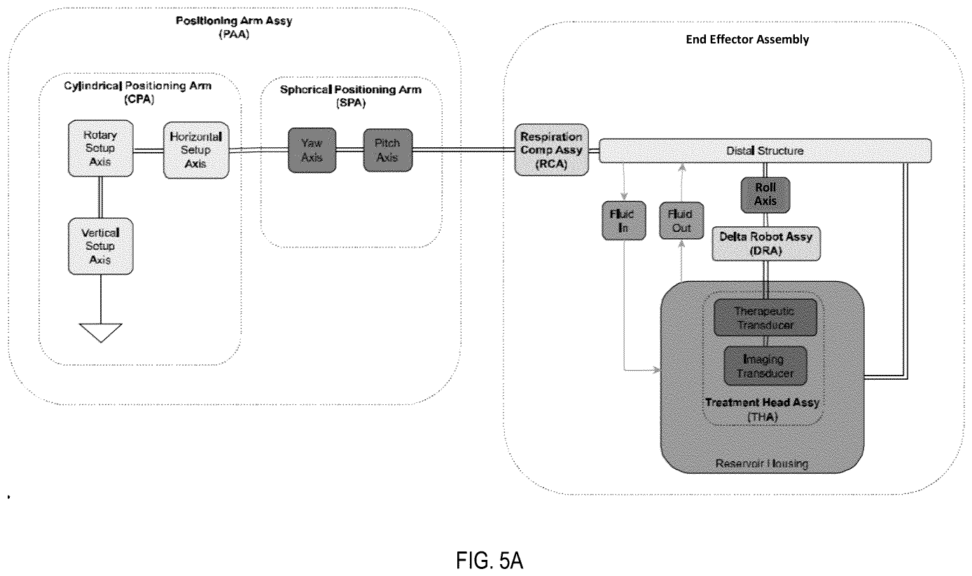

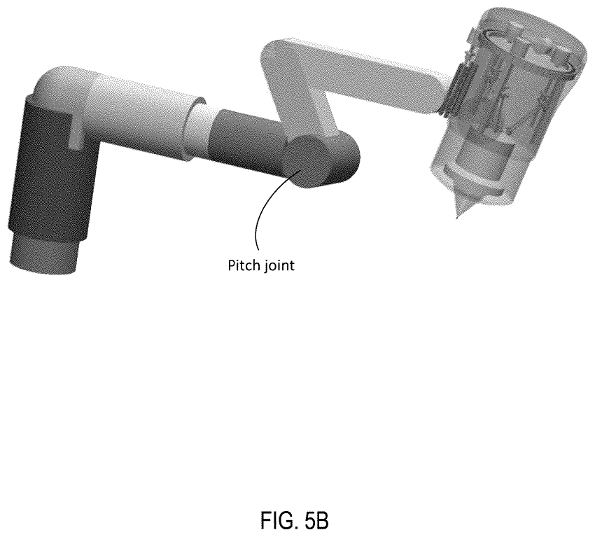

FIGS. 5A and 5B depict a second example of a HIFU system, including a positioning arm assembly and an end effector assembly, according to embodiments. FIG. 5A schematically depicts the components of the HIFU system, and FIG. 5B provides a drawing of the positioning arm assembly and the end effector assembly. The HIFU system depicted in FIGS. 5A and 5B can be structurally and/or functionally similar to other histotripsy systems described herein. For example, similar to the HIFU system depicted in FIGS. 4A and 4B, the HIFU system depicted in FIGS. 5A and 5B can include a positioning arm assembly that has a cylindrical positioning arm. The positioning arm assembly can also include a spherical positioning arm having one or more joints that provide motion about a yaw axis and a pitch axis. The HIFU system also includes an end effector assembly, similar to that described with respect to FIGS. 4A and 4B. While FIGS. 5A and 5B depict a delta robot assembly as an example of a treatment transducer positioning device, it can be appreciated that end effector assemblies described herein can include any type of treatment transducer positioning device, including a delta robot, cartesian robot, and/or other treatment transducer positioning devices.

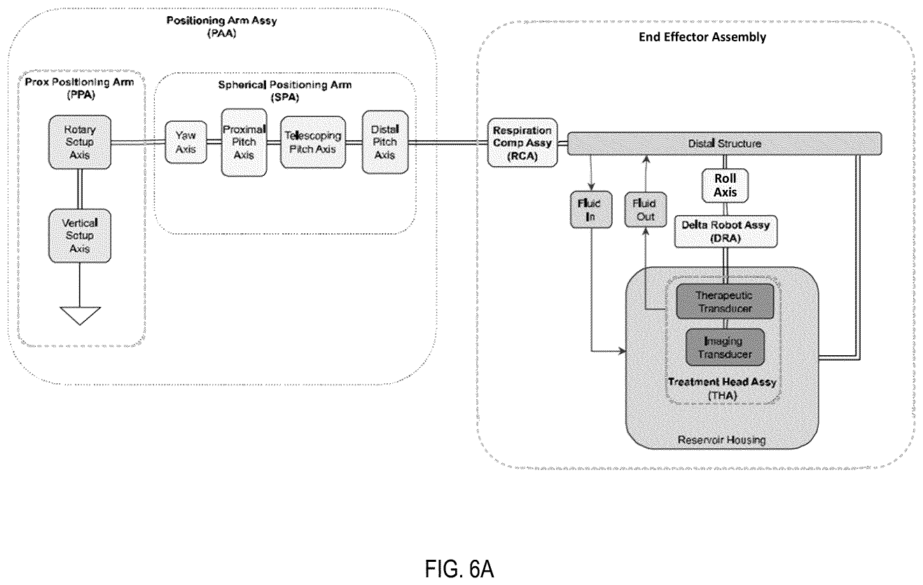

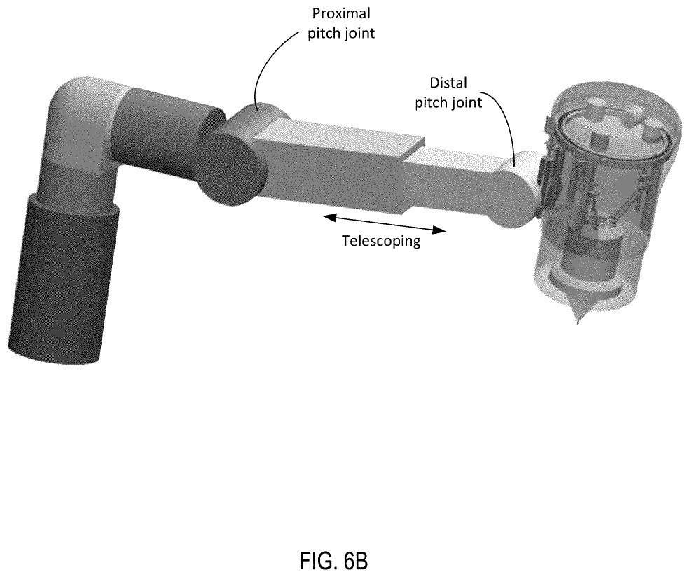

FIGS. 6A and 6B depict a third example of a HIFU system, including a positioning arm assembly and an end effector assembly, according to embodiments. FIG. 6A schematically depicts the components of the HIFU system, and FIG. 6B provides a drawing of the positioning arm assembly and the end effector assembly. The HIFU system depicted in FIGS. 6A and 6B can be structurally and/or functionally similar to other histotripsy systems described herein. For example, similar to the HIFU system depicted in FIGS. 4A and 4B, the HIFU system depicted in FIGS. 6A and 6B can include a positioning arm assembly, but the positioning arm assembly can be configured differently. As depicted in FIG. 6A, the positioning arm assembly can include a proximal positioning arm configured to provide movement about a vertical setup axis (e.g., to allow for vertical height adjustment) and a rotary setup axis (e.g., to allow for rotation). The positioning arm assembly can also include a spherical positioning arm, which can include one or more joints and/or actuators configured to provide movement about a yaw axis, a proximal pitch axis, a telescoping pitch axis and a distal pitch axis. The HIFU system depicted in FIGS. 6A and 6B also includes an end effector assembly, similar to that described with respect to FIGS. 4A and 4B. While FIGS. 6A and 6B depict a delta robot assembly as an example of a treatment transducer positioning device, it can be appreciated that end effector assemblies described herein can include any type of treatment transducer positioning device, including a delta robot, cartesian robot, and/or other treatment transducer positioning devices.

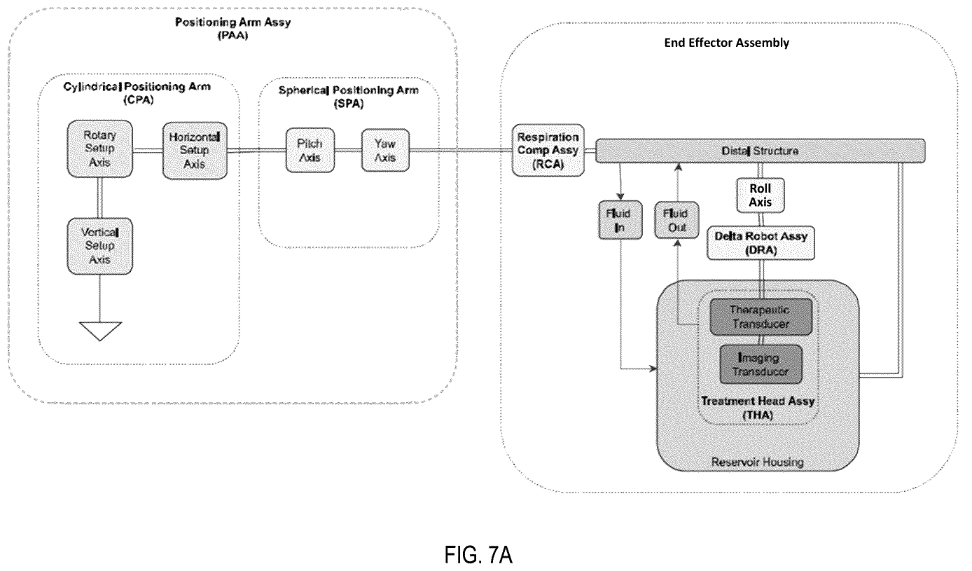

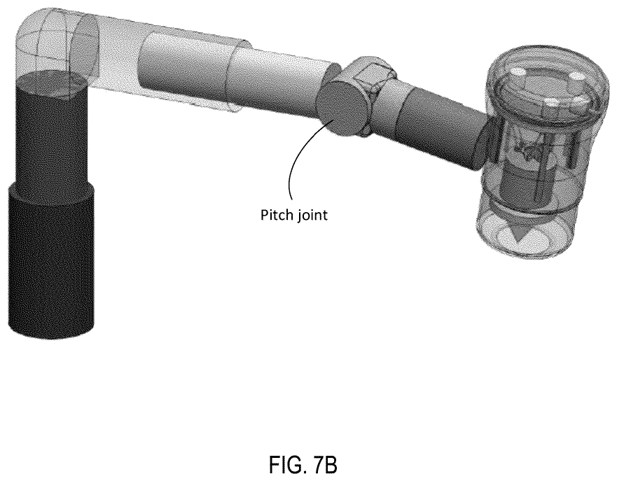

FIGS. 7A and 7B depict a third example of a HIFU system, including a positioning arm assembly and an end effector assembly, according to embodiments. FIG. 7A schematically depicts the components of the HIFU system, and FIG. 7B provides a drawing of the positioning arm assembly and the end effector assembly. The HIFU system depicted in FIGS. 7A and 7B can be structurally and/or functionally similar to other histotripsy systems described herein. For example, similar to the HIFU system depicted in FIGS. 4A and 4B, the HIFU system depicted in FIGS. 7A and 7B can include a positioning arm assembly that has a cylindrical positioning arm. The positioning arm assembly can also include a spherical positioning arm having one or more joints that provide motion about a yaw axis and a pitch axis. The HIFU system also includes an end effector assembly, similar to that described with respect to FIGS. 4A and 4B. While FIGS. 7A and 7B depict a delta robot assembly as an example of a treatment transducer positioning device, it can be appreciated that end effector assemblies described herein can include any type of treatment transducer positioning device, including a delta robot, cartesian robot, and/or other treatment transducer positioning devices.

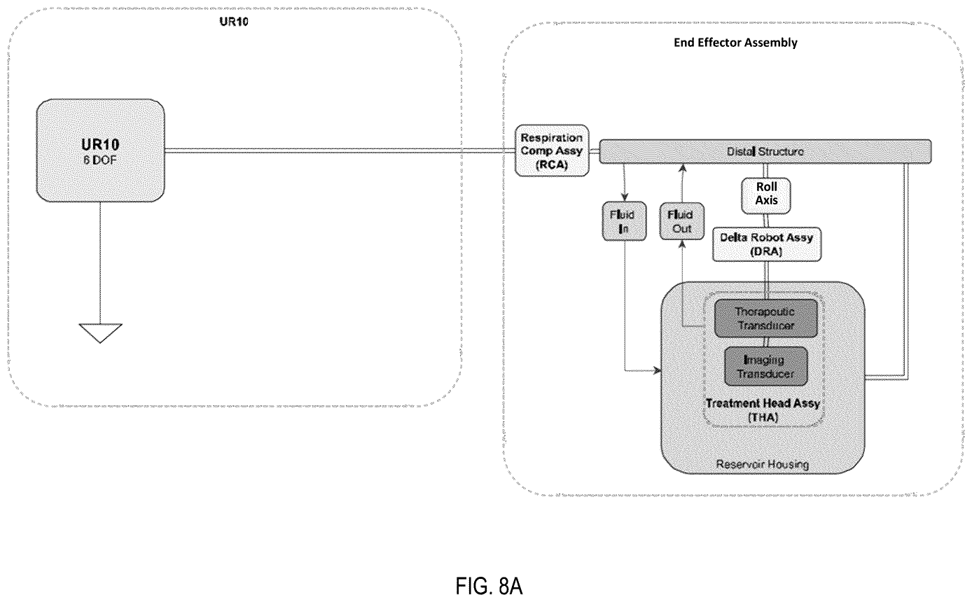

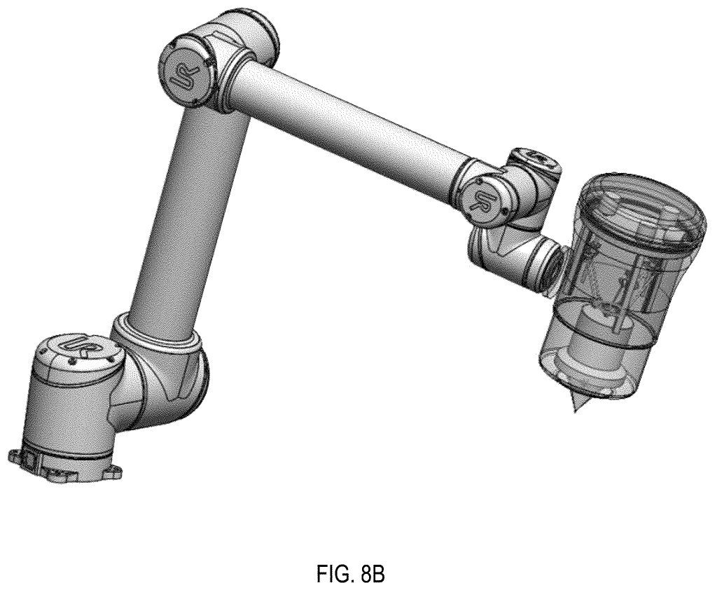

FIGS. 8A and 8B depict a fourth example of a HIFU system, including a positioning arm assembly and an end effector assembly, according to embodiments. FIG. 8A schematically depicts the components of the HIFU system, and FIG. 8B provides a drawing of the positioning arm assembly and the end effector assembly. The HIFU system depicted in FIGS. 8A and 8B can be structurally and/or functionally similar to other histotripsy systems described herein. For example, the HIFU system depicted in FIGS. 8A and 8B can include a robotic arm having a plurality of joints and segments that provide motion in a plurality of DOFs. In an embodiment, the robotic arm can be configured to provide six DOFs. In an embodiment, the robotic arm can have a base joint, a shoulder joint, an elbow joint, and three wrist joints (e.g., roll, pitch, and yaw). The HIFU system also includes an end effector assembly, similar to that described with respect to FIGS. 4A and 4B. While FIGS. 8A and 8B depict a delta robot assembly as an example of a treatment transducer positioning device, it can be appreciated that end effector assemblies described herein can include any type of treatment transducer positioning device, including a delta robot, cartesian robot, and/or other treatment transducer positioning devices.

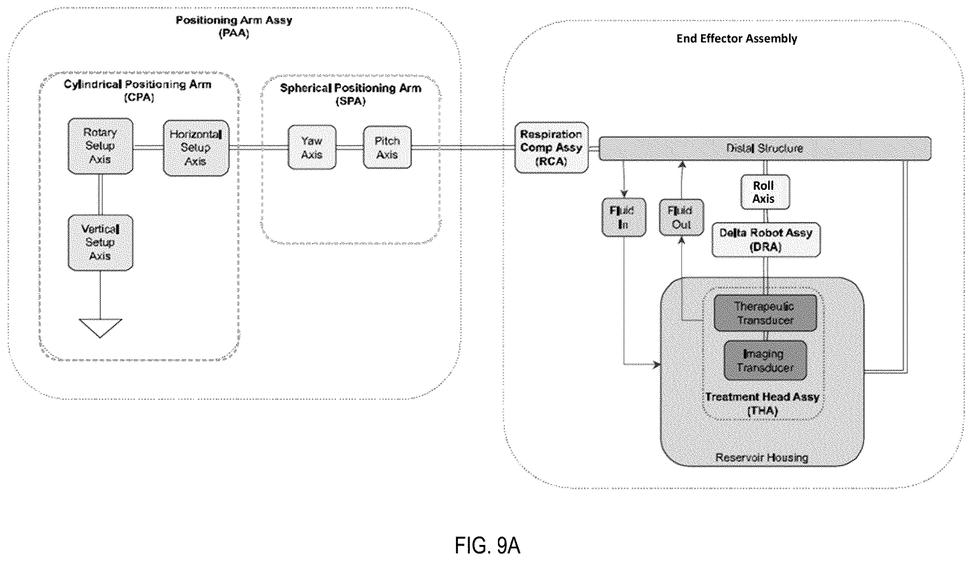

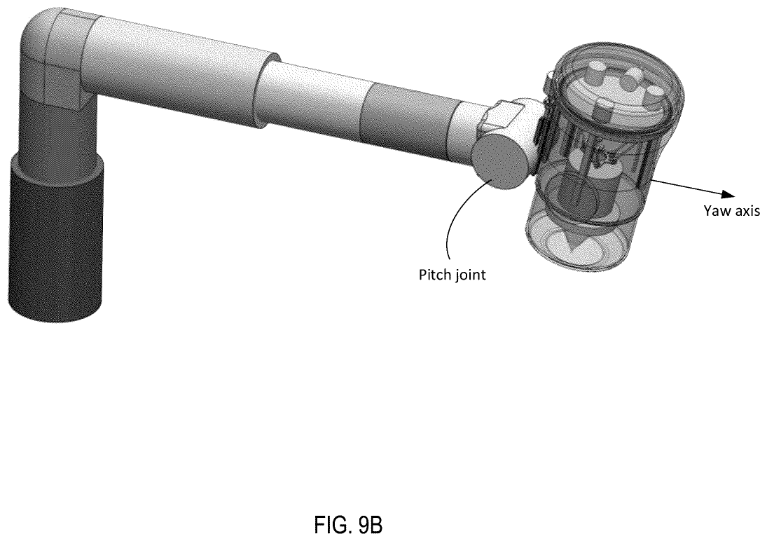

FIGS. 9A and 9B depict a fourth example of a HIFU system, including a positioning arm assembly and an end effector assembly, according to embodiments. FIG. 9A schematically depicts the components of the HIFU system, and FIG. 9B provides a drawing of the positioning arm assembly and the end effector assembly. The HIFU system depicted in FIGS. 9A and 9B can be structurally and/or functionally similar to other histotripsy systems described herein. For example, similar to the HIFU system depicted in FIGS. 4A and 4B, the HIFU system depicted in FIGS. 9A and 9B can include a positioning arm assembly that has a cylindrical positioning arm. The positioning arm assembly can also include a spherical positioning arm having one or more joints that provide motion about a yaw axis and a pitch axis. The HIFU system also includes an end effector assembly, similar to that described with respect to FIGS. 4A and 4B. While FIGS. 9A and 9B depict a delta robot assembly as an example of a treatment transducer positioning device, it can be appreciated that end effector assemblies described herein can include any type of treatment transducer positioning device, including a delta robot, cartesian robot, and/or other treatment transducer positioning devices.

In some embodiments, the example systems described herein, such as those depicted in FIGS. 4A-9B, can be used for transcutaneous or extracorporeal applications, e.g., where the treatment head assembly is disposed outside of a body lumen, such as above the abdominal region and/or thoracic region. With transcutaneous or extracorporeal applications, it can be desirable to have a positioning arm (e.g., such as those in any of the positioning arm assemblies described herein) that includes at least three degrees of freedom. For example, systems configured for transcutaneous or extracorporeal applications can include a positioning arm that has a first joint that provides for rotation of the end effector assembly about a rotary setup axis (as described above), a second joint that provides for translation or insertion of the end effector assembly about a horizontal setup axis (as described above), and a third joint that provides for rotation in pitch of the end effector assembly (e.g., about a gimbal axis as described above). Therefore, while the examples provided in FIGS. 4A-9B include positioning arms that enable more than three degrees of freedom, such may not be required. In some embodiments, a positioning arm as described herein can include three degrees of freedom up to and inclusive of six or more degrees of freedom.

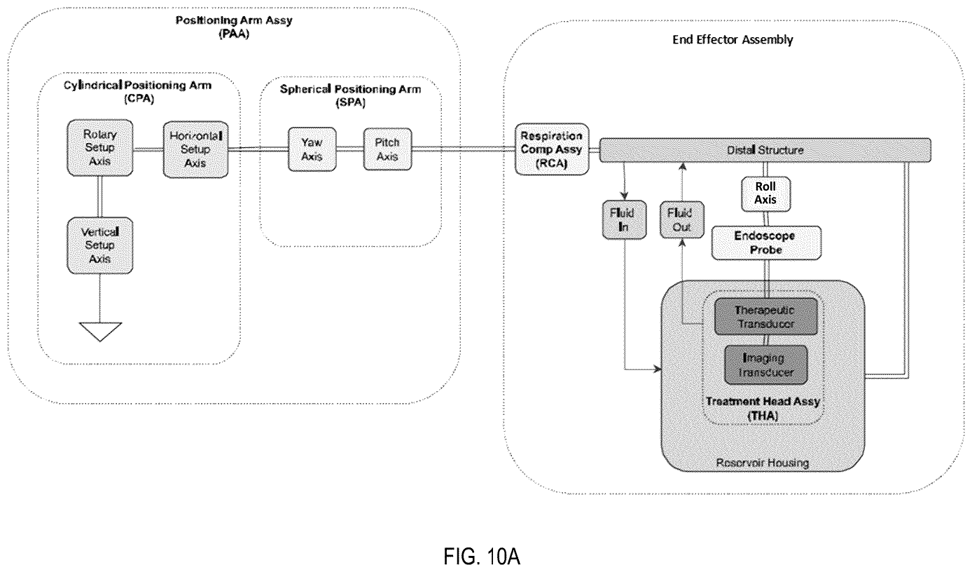

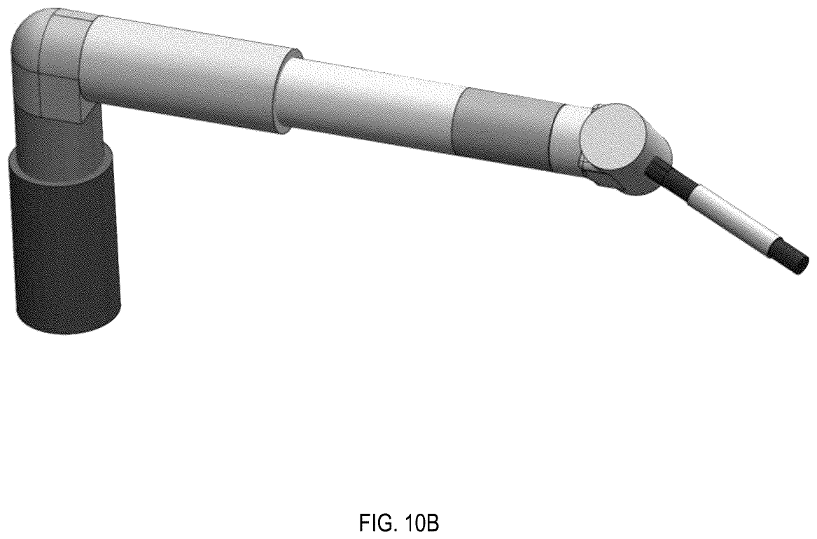

Alternatively, or additionally, HIFU systems described herein can include a probe or catheter (e.g., an endoscopic probe), a needle or other percutaneous device, etc. These devices can be configured to deliver the treatment head assembly into a patient's body, e.g., for endoluminal (e.g., transrectal, transvaginal, or transoral) and/or percutaneous applications of HIFU treatment (e.g., such as BH). With such applications, it can be desirable to have a positioning arm (e.g., such as those in any of the positioning arm assemblies described herein) that includes at least three degrees of freedom. For example, systems configured for endoluminal or percutaneous applications can include a positioning arm that has a first joint that provides for rotation of the end effector assembly and/or treatment head assembly (e.g., about a longitudinal axis of the probe or device), a second joint that provides for translation or insertion of the end effector assembly and/or treatment head assembly (e.g., into a lumen, or in a direction along a longitudinal axis of the probe or device), and a third joint that provides for rotation in pitch of the end effector assembly and/or treatment head assembly (e.g., about an axis near a distal end of the probe or device, such as a wrist joint). In some embodiments, a positioning arm as described herein can include three degrees of freedom up to and inclusive of six or more degrees of freedom. Also, it can be appreciated that with a probe, catheter, or other internally inserted device, that such devices can be configured to have a single degree or freedom (e.g., bending in one direction), or multiple degrees of freedom (e.g., two or more). For example, in some embodiments, a probe or catheter can be flexible. For example, the probe or catheter can be a flexible component that can be navigated or steered to a target location, e.g., for delivering HIFU treatment. FIGS. 10A and 10B depict an example of a robotic system that can be suitable for endoluminal applications.

FIGS. 10A and 10B depict a fifth example of a HIFU system, including a positioning arm assembly and an end effector assembly, according to embodiments. FIG. 10A schematically depicts the components of the HIFU system, and FIG. 10B provides a drawing of the positioning arm assembly and the end effector assembly. The HIFU system depicted in FIGS. 10A and 10B can be structurally and/or functionally similar to other histotripsy systems described herein, but include an endoscopic probe, which can support the treatment head assembly within a body of a patient (e.g., within a body lumen). For example, the treatment head assembly (including a treatment device such as a treatment transducer array, e.g., treatment transducer array 226) can be disposed on a distal end portion or region of the probe. In such embodiments, the positioning device for moving the treatment device (e.g., transducer array positioning device 2618) can be configured to move the distal end portion of the endoluminal probe and/or catheter in at least three degrees-of-freedom including, for example, translation along an insertion axis and rotation about two axes (e.g., a roll axis and a pitch axis).

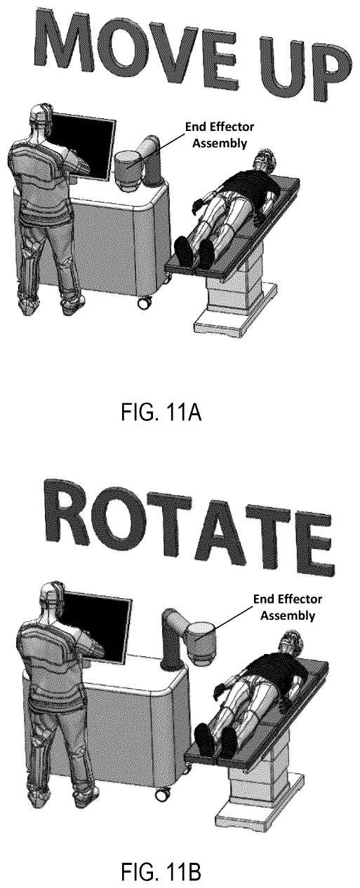

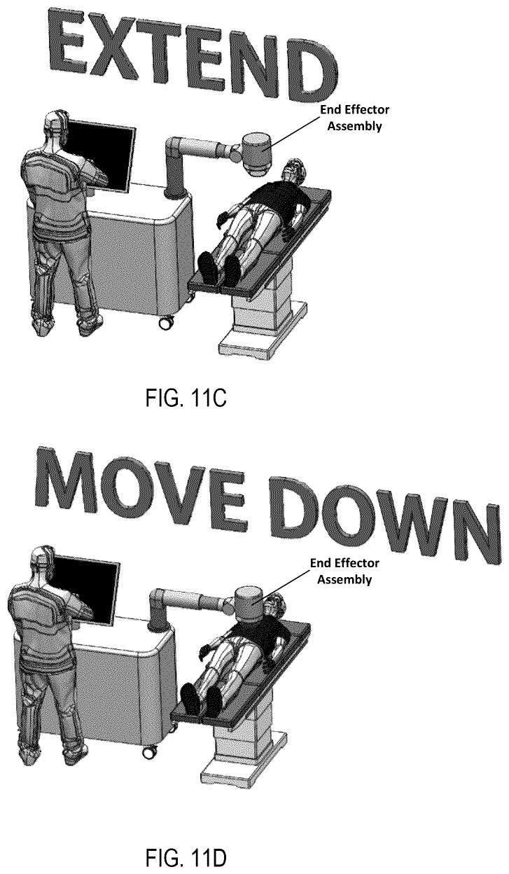

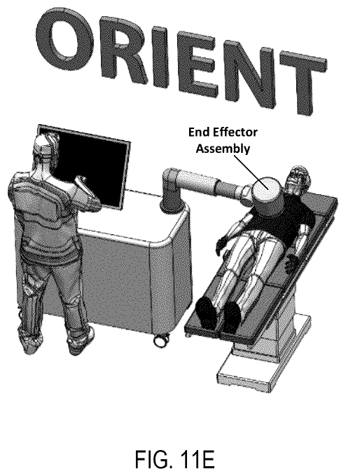

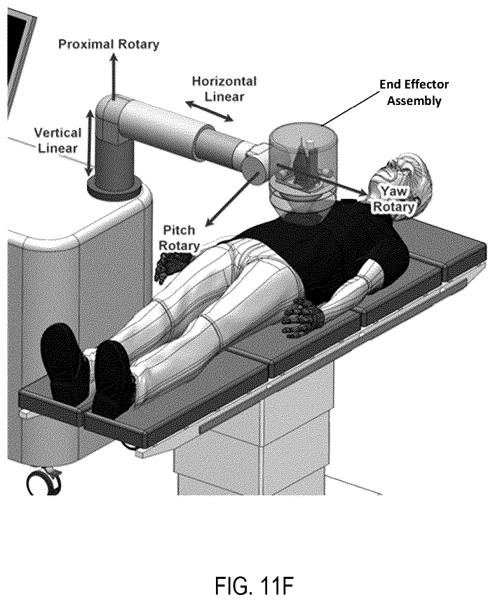

In operation, the robotic arms of the HIFU systems described herein can be configured to position a treatment head assembly for applying BH to a target area of a patient body, as shown in FIGS. 11A-11E. For example, the robotic arms can be configured to vertically raise (e.g., move up) an end effector assembly (including a treatment head assembly) (FIG. 11A), rotate the end effector assembly toward a patient body (FIG. 11B), extend the end effector assembly out toward the patient body (FIG. 11C), vertically lower (e.g., move down) the end effector assembly toward the patient body (FIG. 11D), and orient the end effector assembly about a target area (FIG. 11E). FIG. 11F depicts an example HIFU system, with the DOFs of the robotic arm depicted, showing how the robotic arm can enable various movements of the end effector assembly. The systems depicted in FIGS. 11A-11E can include components that are structurally and/or functionally similar to other HIFU systems, robotic systems, positioning arms, and/or components thereof described herein.

As described above, the treatment head assembly can include a transducer array (e.g., therapeutic transducer). The transducer array can be configured to apply BH treatment to a target area. While applying the BH treatment, the transducer array can be configured to translate linearly about one or more axes and/or rotate about one or more axes, e.g., to change a focal point or focal volume of the treatment. Movements of the focal point or focal volume of the treatment can enable BH to be applied to both a larger treatment volume and more discreetly defined treatment shape. FIGS. 12B-12D depict example movement of a treatment transducer array as it applies BH treatment. FIG. 12A provides a detailed view of the treatment head assembly, with labels showing the DOFs of the treatment transducer array (e.g., therapeutic transducer).

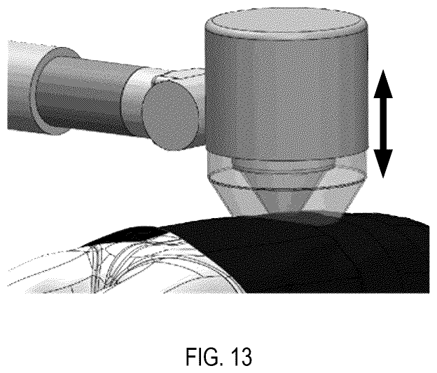

In some embodiments, robotic devices described herein can also provide respiration compensation, e.g., movements that compensate for movement of the patient chest wall or abdominal wall, e.g., as a result of respiration. FIG. 13 illustrates the vertical height changes that can occur for respiration compensation. In some embodiments the histotripsy device 120 can include one or more sensor(s) at the interaction point to monitor the coupling of the device (e.g., interface 228 or other coupling interfaces as described herein) to the patient during respiratory compensation or other types of motion compensation (e.g., bowel movement, heart movements, gross movements, etc.).

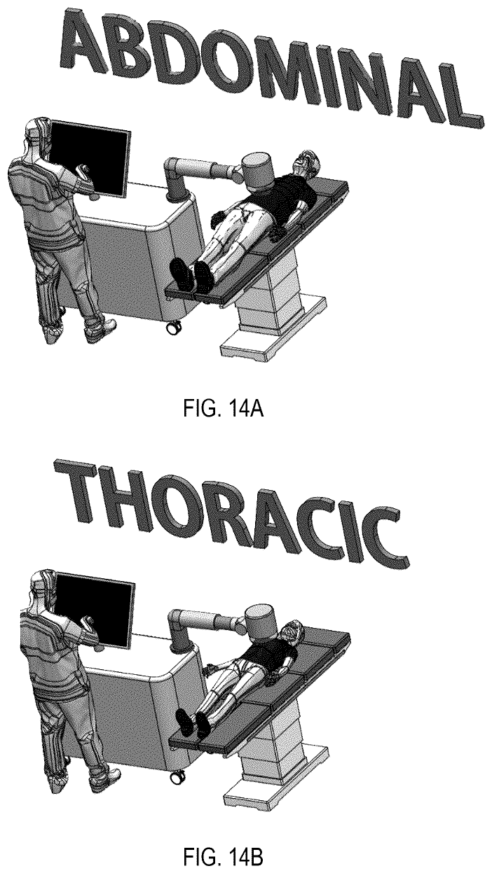

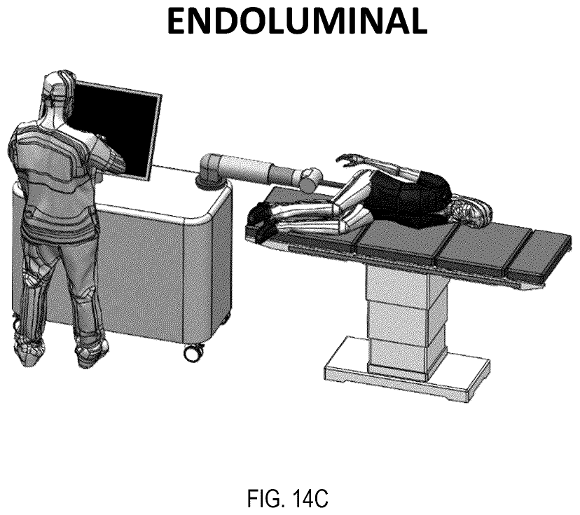

FIGS. 14A-14C depict different use cases of HIFU systems described herein. For example, the HIFU systems described herein can be configured to extracorporeally treat (e.g., liquefy) selected tissue types, e.g., in the cranial, cervical, thoracic, abdominal, and/or pelvic regions. For such treatments, the treatment probe can be placed abdominally and/or thoracically, as shown in FIGS. 14A and 14B, respectively. In some embodiments, the end effector assemblies of HIFU systems as described herein can be configured to placed in other regions of a patient, e.g., cervically for neck, cranially for head, etc. In some embodiments, the HIFU systems can be configured to endoluminally treat selected tissue types, e.g., through transoral, transvaginal or transrectal insertion of the treatment head. FIG. 14C shows an endoluminal approach.

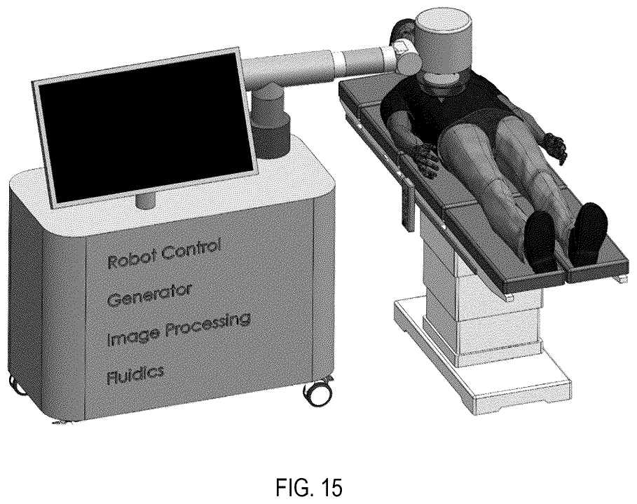

FIG. 15 depicts a cart of the HIFU systems described herein, according to embodiments. The cart can be configured to include one or more input and output devices, such as a touchscreen display, keyboard, mouse, joystick, etc., which can be used to receive inputs from a medical professional and/or present outputs to the medical professional. The cart can be configured to provide control of the robotic arm and end effector assembly, generate waveforms for delivering HIFU treatment (e.g., BH), perform image processing (e.g., for pre-operative positioning, procedure guidance, etc.), and/or control fluid delivery or removal from the reservoir or bladder. The cart can be configured to stow the robotic arm when transporting the system to alternate locations. In some embodiments, the cart and the robotic arm can include a portion that is covered by sterile draping. Such may be implemented for percutaneous, endoluminal, and/or other minimally invasive or invasive procedures. The sterile draping can be configured to cover any non-sterile portions of the histotripsy system to maintain a sterile environment during treatment. In some embodiments, the sterile portions of the histotripsy system (end effector assembly, components thereof, and/or other components attached thereto (such as probes, biopsy needles, etc.)) can be configured to be removably coupled to draped portions of the system (positioning arm or other portions of the robotic system, cart, etc.) via sterile connection mechanisms or through openings in the sterile draping. In some embodiments, the sterile draping can be configured to cover at least a portion of the positioning arm and/or the cart. In some embodiments, the sterile draping can be configured to cover an entirety of the positioning arm, while not covering the end effector assembly. In some embodiments, the sterile draping can be configured to cover at least a portion of the positioning arm and/or end effector assembly. The sterile draping can be designed to be flexible, e.g., to accommodate dynamic movement of joints and/or segments of the robotic system without compromising the sterile barrier.

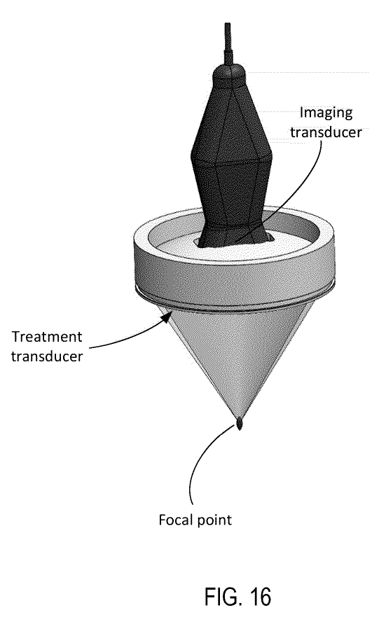

FIG. 16 depicts an example of a HIFU transducer assembly or treatment head assembly, including a treatment transducer and an imaging transducer, according to embodiments. The treatment head assembly can be functionally and/or structurally similar to other treatment head assemblies or histotripsy devices described herein. For example, the treatment transducer can be structurally and/or functionally similar to treatment transducer arrays described herein, including, for example treatment transducer array 226. The imaging transducer can be structurally and/or functionally similar to other imaging transducers described herein, including, for example imaging transducer 224. The treatment transducer can be focusing to a focal point or focal volume, as labeled in FIG. 16. The imaging transducer can be positioned toward or at a center of the treatment head assembly and can be configured to capture a view of the anatomical region that includes the focal point.

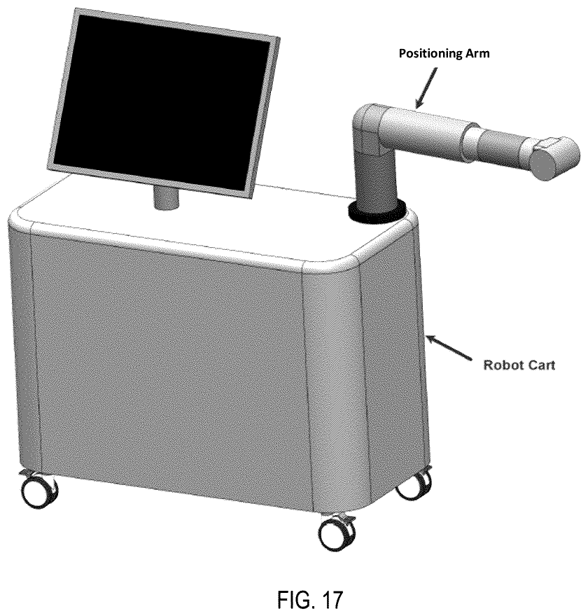

FIG. 17 depicts a robotic arm of a robotic surgical system coupled to a robot cart, according to an embodiment. The cart can be configured to include one or more input and output devices, such as a touchscreen display, keyboard, mouse, joystick, etc., which can be used to receive inputs from a medical professional and/or present outputs to the medical professional. The cart can be configured to provide control of the robotic arm, generate waveforms for delivering BH treatment, perform image processing (e.g., for pre-operative positioning, procedure guidance, etc.), and/or control fluid delivery or removal from the reservoir or bladder. The cart is coupled (e.g., attached) to a robotic arm (e.g., universal positioning arm (UPA)). The robotic arm is configured to accept various end effectors or end effector assemblies (e.g., transducer arrays, treatment devices, etc.) as described herein.

FIGS. 18A-18B depict another example robotic arm of a robotic surgical system configured to control movements of a distal structure of a HIFU system, according to embodiments. FIG. 18A schematically depicts the components of the positioning arm, and FIG. 18B depicts a drawing of the positioning arm and the axes of movement. The positioning arm is as 5-DOF (five degree-of-freedom) arm and is configured to operate in two linear axes (e.g., vertical, horizontal, etc.) and three rotary axes (e.g., shoulder, yaw, pitch). In some embodiments, the linear axes are motorized by ball screw drives. In some embodiments, the rotary axes are motorized with harmonic drives. In some embodiments, the axes are user-backdrivable in a power-off fault scenario. In some embodiments, the positioning arm includes a respiration compensation assembly (e.g., RCA). While the RCA is described as compensating for respiration, it can be appreciated that the component can generally compensate for motion from the patient, including other types of motion such as bowel movements, heart movements, involuntary motions, gross movements, etc. Therefore, in embodiments, the RCA can also be referred to as a motion compensation assembly. The RCA can include one or more force or contact sensors (e.g., three force sensors) located between the horizontal and yaw axes configured to measure orthogonal linear forces. The proximal axes (e.g., vertical, shoulder, and horizontal) are servoed to maintain the measured force in a predetermined range about a nominal preload force. In other words, respiratory (or other motion) compensation about each axis can be controlled using one or more servo motors, which can include a closed-loop control system (e.g., via force or contact sensors) that be set to adjust the positioning of a treatment head assembly coupled to the positioning arm to maintain forces within a predefined range of forces. The initial preload force can be set by a user when the positioning arm is positioning relative to the patient (e.g., calibration). While the RCA is depicted in various embodiments described herein (e.g., FIGS. 4A, 5A, 6A, etc.) as a separate component from the positioning arm assembly, it can be appreciated that respiratory (or other motion) compensation can be implemented via the positioning arm alone. For example, the positioning arm can have at least three DOFs that can be configured to compensate for movements of the patient body as a result of respiration (e.g., respiratory movements). In some embodiments, the three DOFs can include a first DOF that adjusts for vertical movements, a second DOF that adjusts for horizontal movements, and rotation about a pitch axis. Alternatively, or additionally, the RCA (or other motion compensation) can be implemented by one or more portions of the micro positioning devices, such as, for example a transducer array positioning device as described herein.

FIGS. 19A-19B depict a sixth example of a HIFU system, including a positioning arm (e.g., functionally and/or structurally similar to the positioning arm described in reference to FIGS. 18A-18B) and a transcutaneous end effector (TCEE), according to embodiments. The HIFU system of FIGS. 19A-19B is a transcutaneous delivery system (TCDS). The HIFU system depicted in FIGS. 19A and 19B can be structurally and/or functionally similar to other histotripsy systems described herein. FIG. 19A schematically depicts the components of the TCDS while FIG. 19B depicts the DOFs of the end effector of the TCDS according to an embodiment. The TCDS is configured to be used in a thoracic (e.g., breast), cervical (e.g., thyroid), and/or an abdominal (e.g., liver, kidney, pancreas) patient setup in the ventral cavity as seen in FIGS. 14A and 14B.

The TCEE is a robotic subassembly that can attach to the positioning arm (e.g., structurally and/or functionally similar to other positioning arms or positioning arm assemblies described herein, including, for example, positioning arm 2617). The connection between the TCEE and the positioning arm can include fluid lines, transducer signals, power signals, data signals, and/or the like. The TCEE robotically delivers a transcutaneous treatment head (TCTH) which includes a therapeutic transducer and/or an imaging transducer. In some embodiments, the TCEE has four DOFs to position and orient the TCTH. The DOFs can include three orthogonal linear axes (e.g., x, y, z, cartesian, etc.) and a roll axis distal to the orthogonal linear axes, as seen in FIG. 19B.

As seen in FIG. 20, the TCEE includes a cartesian robot mounted in a fixed housing. The TCTH is attached to the distal end of the cartesian robot. In some embodiments, the TCTH can roll along a roll axis relative to the cartesian robot. In some embodiments, the TCEE includes a single-use reservoir attached to the fixed housing. The reservoir can be filled with an account coupling medium such as water and/or the like. The cartesian robot is configured to move relative to the positioning arm as seen in FIGS. 12B-12D. While FIGS. 19A and 19B refer to a cartesian robot assembly as an example of a treatment transducer positioning device, it can be appreciated that end effector assemblies described herein can include any type of treatment transducer positioning device, including a cartesian robot and/or other treatment transducer positioning devices. Moreover, it can be appreciated that the TCEE can have less than four DOFs or more than four DOFs.

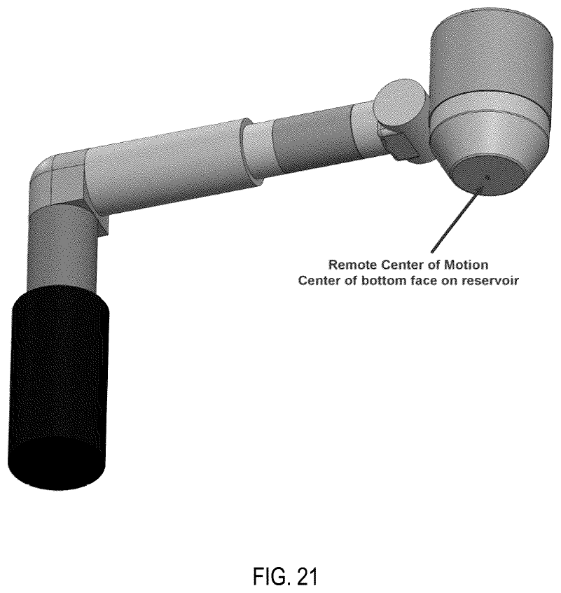

The TCDS, during operation, can include a positioning clutch mode. The positioning clutch mode is user controlled by pressing a clutch button on or near a pitch axis. During the positioning clutch mode, three DOFs are moveable including a vertical (e.g., up or down) (e.g., counterbalanced to float in the vertical direction), shoulder (e.g., rotate) (e.g., no gravity load), and/or horizontal (e.g., extend or retract) (e.g., no gravity load). The TCDS can also include an orientation clutch mode. The orientation clutch mode is user controlled by pressing a clutch button on or near the TCEE. In some embodiments, all five DOFs of the positioning arm are software controlled (e.g., via a processor, such as, for example, processor 212) to rotate about a point at the center of the reservoir, as seen in FIG. 21.

FIG. 21 depicts a center of motion of a robotic arm, according to an embodiment.

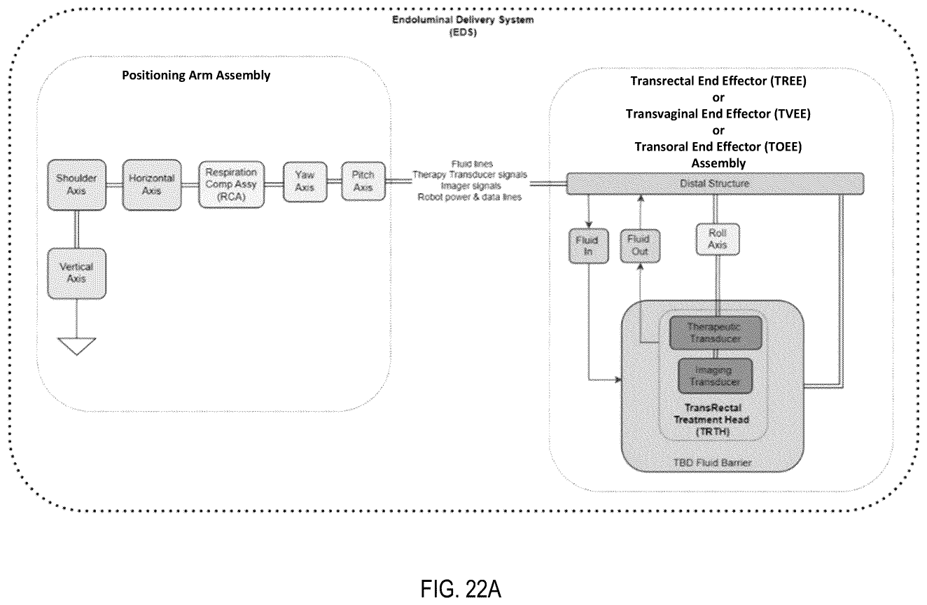

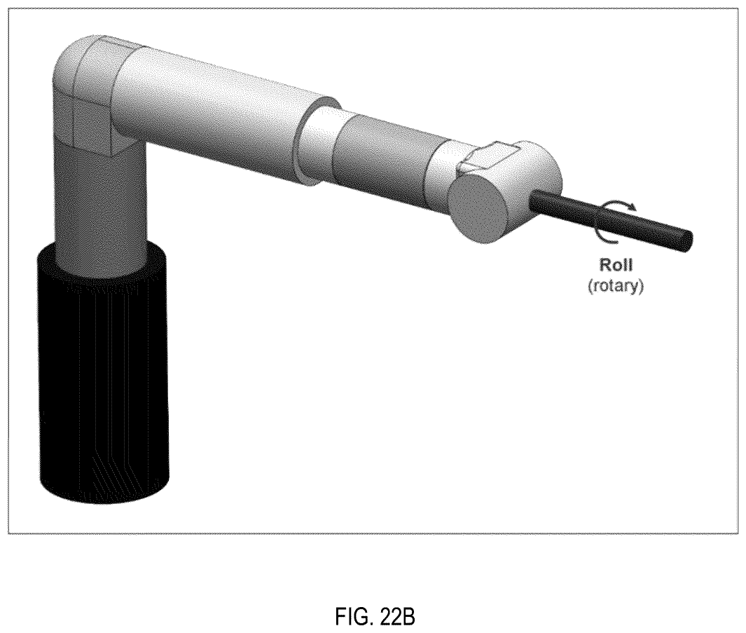

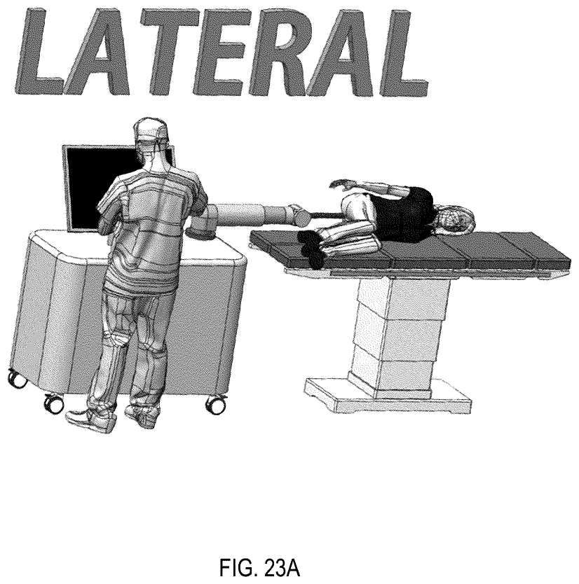

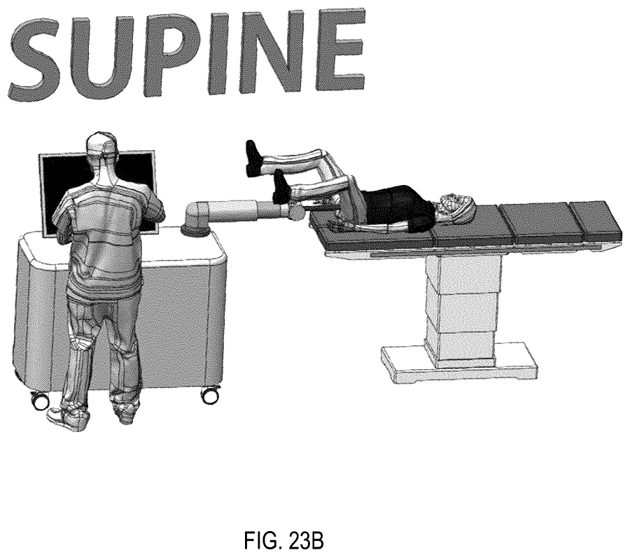

FIGS. 22A-22B depict a seventh example of a HIFU system, including a positioning arm (e.g., functionally and/or structurally similar to other positioning arms described herein, including the positioning arm 2617 and/or the positioning arm described in reference to FIGS. 18A-18B) and an end effector that can include a transoral end effector (TOEE), a transrectal end effector (TREE), and/or a transvaginal end effector (TVEE), according to embodiments. The HIFU system of FIGS. 22A-22B is an endoluminal delivery system (EDS). The HIFU system depicted in FIGS. 22A and 22B can be structurally and/or functionally similar to other histotripsy systems described herein. FIG. 22A schematically depicts the components of the EDS while FIG. 22B depicts an image of the EDS. The proximal end of the EDS in FIG. 23B represents the TOEE, TREE, and/or the TVEE. As seen in FIGS. 23A-23B, the EDS is capable of delivering the TREE and/or the TVEE in both the lateral and the supine patient positions.

The end effectors (e.g., TOEE, TREE, and TVEE) can be functionally similar but have different form factors associated with the target anatomy. The end effectors are robotic subassemblies that are coupled to the positioning arm. The coupling can allow for passage of fluid lines, transducer signals, power and data signals. The TOEE is configured to deliver a transoral treatment head (TOTH) which can deliver a therapeutic transducer and/or an imaging transducer. The TREE is configured to deliver a transrectal treatment head (TRTH) which can include a therapeutic transducer and/or an imaging transducer. The TVEE is configured to deliver a transvaginal treatment head (TVTH) which can include a therapeutic transducer and/or an imaging transducer. The TOEE, TREE, and/or TVEE can have one DOF (e.g., roll axis) to position and orient the treatment head. Optionally, the TOEE, TREE, and/or TVEE can have one or more DOFs, including, for example, a second DOF that allows for bending, curving, or flexing of the treatment head, and/or a third DOF that allows for insertion of the treatment head. In some embodiments, the distal end of the TOEE, TREE and/or the TVEE can be similar to what is described in U.S. Pat. No. 11,986,853, titled “Transrectal ultrasound probe for boiling histotripsy ablation of prostate, and associated systems and methods,” filed May 11, 2020, the disclosure of which is incorporated herein by reference.

The EDS can include an EDS clutch mode which is user controlled by a clutch but on or near the TOEE, TREE, or TVEE. In some embodiments, all five DOFs of the positioning arm are positionable in the clutch mode. In some embodiments, the roll DOF (and/or other DOFs such as the insertion DOF, and/or the bending DOF) on the TOEE, TREE, or TVEE is not active in the EDS clutch mode.

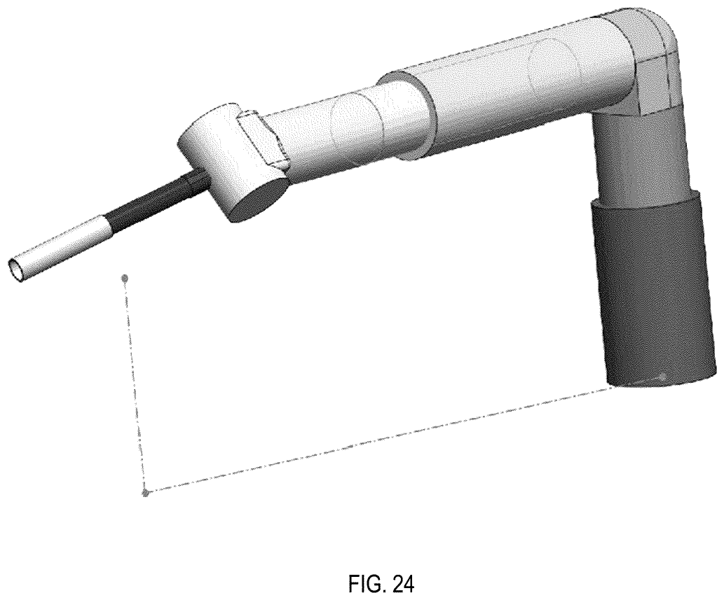

As seen in FIG. 24, the EDS can be orientated during insertion into patient anatomy. The five DOFs of the positioning arm move together to guide an axis of the TOEE, TREE, or TVEE along a trajectory that follows the axis of insertion. The position of the axis of insertion is determined by the position of the vertical, shoulder, and horizontal axes. The orientation axis of insertion is determined by the angles of the yaw and pitch axes. The roll and/or flex axis in the TOEE, TREE, or TVEE can move independently. In some embodiments, a coupling medium (e.g., interface) is included during the insertion of the TOEE, TREE, or TVEE to provide an interface between the transducer array (or a distal end portion of a probe and/or catheter carrying the transducer array) and tissue in the endoluminal region of the patient P. The coupling medium can be a flexible bag, balloon, container, outer reservoir, and/or the like of an interfacing fluid. In some embodiments, the coupling medium can include fluids (e.g., degassed fluids) that are pumped into the target region, without other structure (e.g., reservoir, housing, etc.) for containing the fluids. In such instances, the fluids can be contained, for example, by the walls of the body lumen or adjacent anatomy. In some embodiments, the coupling medium can be pre-filled and/or dynamically pumped into the endoluminal cavity during TOEE, TREE, or TVEE insertion.

An eighth example of a HIFU system can include a positioning arm and a percutaneous end effector (PCEE) or instrument, according to embodiments. The HIFU system can be structurally and/or functionally similar to other histotripsy systems described herein. For example, the positioning arm can be the same as the positioning arm depicted in FIGS. 18A and 18B. The PCEE is configured to insert a percutaneous treatment head (PCTH) into an anatomical structure of a patient P to deliver a therapeutic transducer and/or an imaging transducer. In some embodiments, a portion of the PCEE can include a tool tip such as, for example, a needle or sharp tip configured to penetrate through skin of a patient. In some embodiments, the PCEE can have one DOF, or more DOFs. For example, the PCEE can have four DOFs to position and orient the treatment head. The DOFs can include three orthogonal linear axes (e.g., x, y, z, cartesian, etc.) and a roll axis distal to the orthogonal linear axes, as shown in FIG. 19B with the TCEE. Alternatively, in other embodiments, the PCEE can have three DOFs, such as, for example, insertion, roll, and pitch. Still alternatively, in other embodiments, the PCEE can have two DOFs, such as, for example, insertion and roll.

Further examples of histotripsy systems are provided in International Patent Application No. PCT/US25/25214, titled “HISTOTRIPSY SYSTEMS INCLUDING STEERABLE ARRAYS, AND DEVICES AND METHODS THEREOF,” filed Apr. 17, 2025, International Patent Application No. PCT/IB2025/051100, titled “SYSTEMS, DEVICES, AND METHODS FOR NON-INVASIVE TREATMENT OF TISSUE USING BOILING HISTOTRIPSY,” filed Jan. 31, 2025, the disclosure of each of which is incorporated herein by reference in its entirety.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, and/or method described herein. In addition, any combination of two or more such features, systems, articles, materials, and/or methods, if such features, systems, articles, materials, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

As used herein, the terms “about” and/or “approximately” when used in conjunction with numerical values and/or ranges generally refer to those numerical values and/or ranges near to a recited numerical value and/or range. In some instances, the terms “about” and “approximately” may mean within ±10% of the recited value. For example, in some instances, “about 100 [units]” may mean within ±10% of 100 (e.g., from 90 to 110). The terms “about” and “approximately” may be used interchangeably.

Some embodiments described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

The specific examples and descriptions herein are exemplary in nature and embodiments may be developed by those skilled in the art based on the material taught herein without departing from the scope of the present invention, which is limited only by the attached claims.

1. An apparatus, comprising:
an end effector assembly including:
a treatment device including an array of ultrasound transducers configured to generate pulses of pressure waves that are configured to travel through an interfacing fluid and into tissue to induce boiling histotripsy at a focal volume, a positioning device configured to move the treatment device in at least three degrees-of-freedom to cause the focal volume to move throughout a three-dimensional (3D) target area of a patient to treat the 3D target area using boiling histotripsy, and
a percutaneous treatment head, the treatment device being disposed on the percutaneous treatment head, the percutaneous treatment head configured to be inserted into skin of the patient to position the treatment device near the 3D target area, the positioning device is configured to provide movement of the percutaneous treatment head in three degrees-of-freedom including: translation along an insertion axis and rotation about two axes; and
a robotic positioning arm coupled to the end effector assembly, the robotic positioning arm including a plurality of joints and segments that are configured to move the end effector assembly in at least three degrees-of-freedom to position the treatment device near the 3D target area and to compensate for gross movements such that the treatment device can treat the 3D target area.

2. The apparatus of claim 1, wherein the percutaneous treatment head includes a probe and/or catheter with a needle or sharp tip configured to penetrate through the skin of the patient.

3. The apparatus of claim 1, wherein the end effector assembly includes a container configured to contain an interfacing fluid, the container including a surface positionable adjacent to a skin surface of the patient.

4. The apparatus of claim 3, wherein the end effector assembly includes one or more sensors at an interaction point between the container and the skin surface to monitor a coupling of the end effector assembly to the patient to compensate for respiratory movements.

5. An apparatus, comprising:
an end effector assembly including:
a treatment device including an array of ultrasound transducers configured to generate pulses of pressure waves that are configured to travel through an interfacing fluid and into tissue to induce boiling histotripsy at a focal volume,
a positioning device configured to move the treatment device in at least three degrees-of-freedom to cause the focal volume to move throughout a three-dimensional (3D) target area of a patient to treat the 3D target area using boiling histotripsy,
a container configured to contain an interfacing fluid, the container including a surface positionable adjacent to a skin surface of the patient, and
one or more sensors at an interaction point between the container and the skin surface to monitor a coupling of the end effector assembly to the patient to compensate for respiratory movements; and
a robotic positioning arm coupled to the end effector assembly, the robotic positioning arm including a plurality of joints and segments that are configured to move the end effector assembly in at least three degrees-of-freedom to position the treatment device near the 3D target area and to compensate for gross movements such that the treatment device can treat the 3D target area.

6. The apparatus of claim 5, wherein the end effector assembly includes a percutaneous treatment head, the treatment device being disposed on the percutaneous treatment head, the percutaneous treatment head configured to be inserted into skin of the patient to position the treatment device near the 3D target area.

7. The apparatus of claim 6, wherein the percutaneous treatment head includes a probe and/or catheter with a needle or sharp tip configured to penetrate through the skin of the patient.
